(19) United States
(12) Patent Application Publication
(10) Pub. No.: US 2009/0026930 A1
(43) Pub. Date: Jan. 29, 2009

(54) Aromatic compound, organic light-emitting diode including organic layer including the aromatic compound, and method of manufacturing the organic light-emitting diode

(76) Inventors: Dong-woo Shin, Yongin-si (KR); Byoung-ki Choi, Yongin-si (KR); Tae-yong Noh, Yongin-si (KR); O-hyun Kwon, Yongin-si (KR); Myeong-suk Kim, Yongin-si (KR); Yu-jin Kim, Yongin-si (KR); Eun-sil Han, Yongin-si (KR); Woon-jung Paek, Yongin-si (KR)

Correspondence Address: ROBERT E. BUSHNELL & LAW FIRM, 2029 K STREET NW, SUITE 600, WASHINGTON, DC, 20006-1004, US

(21) Appl. No.: 12/078,334

(22) Filed: Mar. 28, 2008

(30) Foreign Application Priority Data
Jul. 24, 2007 (KR) 10-2007-0074126

(51) Int. Cl. H01J 1/63; C07D 213/02; C07D 333/02; B05D 5/12; C07C 211/54; C07C 13/28

(52) U.S. Cl. 313/504; 546/285; 549/80; 564/426; 585/26; 427/66

(57) ABSTRACT

An aromatic compound represented by Formula 1 below and an organic light-emitting diode including the same:

M₁-(B)ₙ-M₂ (1)

The aromatic compound has excellent thermal stability and emission characteristics. Thus, the organic light-emitting diode employing the aromatic compound can exhibit a low driving voltage, high efficiency, and high brightness.

### CROSS-REFERENCE TO RELATED PATENT APPLICATION AND CLAIM OF PRIORITY

This application claims priority from Korean Patent Application No. 10-2007-0074126, filed on Jul. 24, 2007, in the Korean Intellectual Property Office, the disclosure of which is incorporated herein in its entirety by reference.

### BACKGROUND OF THE INVENTION

1. Field of the Invention

The present invention relates to an aromatic compound and an organic light-emitting diode including an organic layer including the aromatic compound. More particularly, the present invention relates to an aromatic compound which has excellent thermal stability and emission characteristics, and when applied to an organic light-emitting diode, can provide a low driving voltage, high efficiency, and high brightness, and an organic light-emitting diode including an organic layer including the aromatic compound.

2. Description of the Related Art

Organic light-emitting diodes (OLEDs) have excellent brightness, driving voltage, and response speed characteristics, and can provide multi-colored images, and thus, extensive research into OLEDs has been conducted.

Generally, OLEDs have a stack structure of anode/organic light-emitting layer/cathode. OLEDs may also have various other structures such as anode/hole injection layer/hole transport layer/light-emitting layer/electron transport layer/electron injection layer/cathode, or anode/hole injection layer/hole transport layer/light-emitting layer/hole blocking layer/electron transport layer/electron injection layer/cathode.

Materials used for OLEDs can be divided into vacuum-depositable materials and solution-coatable materials according to an organic layer formation process. Vacuum-depositable materials must have a vapor pressure of 10⁻⁶ torr or more at 500° C. or less, and may be mainly low molecular weight materials having a weight average molecular weight of 1,200 or less. Solution-coatable materials must have high solubility in solvents so as to form solutions, and include mainly an aromatic or heterocyclic ring.

When manufacturing OLEDs using a vacuum deposition process, manufacturing costs may increase due to use of a vacuum system, and it may be difficult to manufacture high-resolution pixels for natural color displays due to the use of a shadow mask. On the other hand, when manufacturing OLEDs using a solution coating process, e.g., inkjet printing, screen printing, or spin coating, the manufacture of an organic layer is simple, manufacturing costs are low, and a relatively high resolution can be achieved compared to when using a shadow mask.

However, the performance (e.g., thermal stability, color purity) of solution-coatable materials is lower than that of vacuum-depositable materials. Even though the solution-coatable materials have good performance, there arise problems that the materials, when formed into an organic layer, are gradually crystallized to grow into a size corresponding to a visible light wavelength range, and thus, the grown crystals scatter visible light, thereby causing a turbidity phenomenon, and pin holes, etc. may be formed in the organic layer, thereby causing device degradation.

Japanese Patent Laid-Open Publication No. 1999-003782 discloses a two naphthyl-substituted anthracene compound that can be used in a light-emitting layer or a hole injection layer. However, the anthracene compound is poorly soluble in a solvent, and even more, OLEDs employing the anthracene compound have unsatisfactory characteristics.

Therefore, it is necessary to develop a compound capable of forming an organic layer of an organic light-emitting diode, which has excellent thermal stability and emission characteristics irrespective of an organic layer formation process.

### SUMMARY OF THE INVENTION

The present invention provides a compound having excellent thermal stability and emission characteristics, and an organic light-emitting diode including an organic layer including the compound.

According to an aspect of the present invention, there is provided an aromatic compound represented by Formula 1 below:

M₁-(B)ₙ-M₂ <Formula 1>

wherein B is a single bond, a substituted or unsubstituted C₁-C₆₀ alkylene group, a substituted or unsubstituted C₅-C₆₀ cycloalkylene group, a substituted or unsubstituted C₅-C₆₀ heterocycloalkylene group, a substituted or unsubstituted C₅-C₆₀ arylene group, a substituted or unsubstituted C₂-C₆₀ heteroarylene group, or a divalent linking group represented by —N(Z₁)- where Z₁ is hydrogen, a substituted or unsubstituted C₁-C₆₀ alkyl group, or a substituted or unsubstituted C₅-C₆₀ aryl group;

n is an integer of 1 to 10; and

M₁ and M₂ are each independently a terminal group derived from a compound represented by Formula 2 below:

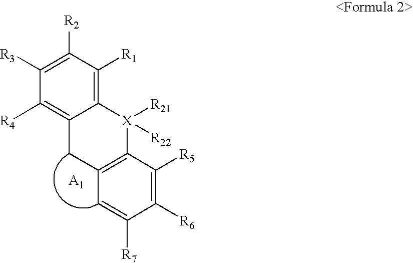

wherein X is a Group XIV element;

R₁, R₂, R₃, R₄, R₅, R₆, R₇, R₂₁, and R₂₂ are each independently hydrogen, halogen, a cyano group, an amino group, a nitro group, a hydroxyl group, a substituted or unsubstituted C₁-C₆₀ alkyl group, a substituted or unsubstituted C₁-C₆₀ alkoxy group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₂-C₆₀ heteroaryl group, a substituted or unsubstituted C₅-C₆₀ arylamino group, a substituted or unsubstituted C₁-C₆₀ alkylamino group, a substituted or unsubstituted C₅-C₆₀ arylsilyl group, or a substituted or unsubstituted C₁-C₆₀ alkylsilyl group, and two or more of R₁, R₂, R₃, R₄, R₅, R₆, R₇, R₂₁, and R₂₂ may be optionally connected or fused together to form a substituted or unsubstituted C₆-C₆₀ aromatic ring or a substituted or unsubstituted C₁-C₆₀ heteroaromatic ring; and

A₁ is a substituted or unsubstituted C₆-C₆₀ aromatic ring or a substituted or unsubstituted C₆-C₆₀ heteroaromatic ring.

Here, at least one hydrogen of the alkylene group, the cycloalkylene group, the heterocycloalkylene group, the arylene group, the heteroarylene group, the alkyl group, the alkoxy group, the alkenyl group, the alkynyl group, the cycloalkyl group, the cycloalkenyl group, the aryl group, and the heteroaryl group may be substituted by a substituent selected from the group consisting of —F; —Cl; —Br; —CN; —NO₂; —NH₂; —OH; a C₁-C₆₀ alkyl group which is unsubstituted or substituted by a C₁-C₆₀ alkoxy group, —F, —Cl, —Br, —CN, —NO₂, —NH₂, or —OH; a C₅-C₆₀ cycloalkyl group which is unsubstituted or substituted by a C₁-C₆₀ alkyl group, a C₁-C₆₀ alkoxy group, —F, —Cl, —Br, —CN, —NO₂, —NH₂, or —OH; a C₅-C₆₀ aryl group which is unsubstituted or substituted by a C₁-C₆₀ alkyl group, a C₁-C₆₀ alkoxy group, —F, —Cl, —Br, —CN, —NO₂, —NH₂, or —OH; and a C₂-C₆₀ heteroaryl group which is unsubstituted or substituted by a C₁-C₆₀ alkyl group, a C₁-C₆₀ alkoxy group, —F, —Cl, —Br, —CN, —NO₂, —NH₂, or —OH.

According to another aspect of the present invention, there is provided an organic light-emitting diode including an organic layer including the above-described aromatic compound.

The aromatic compound has excellent thermal stability and emission characteristics, and thus, the organic light-emitting diode including the organic layer including the same can have a low driving voltage, high efficiency, and high brightness.

### BRIEF DESCRIPTION OF THE DRAWINGS

A more complete appreciation of the invention, and many of the attendant advantages thereof, will be readily apparent as the same becomes better understood by reference to the following detailed description when considered in conjunction with the accompanying drawings in which like reference symbols indicate the same or similar components, wherein:

FIGS. 1A through 1C are schematic sectional views illustrating organic light-emitting diodes according to embodiments of the present invention;

FIG. 2 is a view illustrating the UV absorption and photoluminescence (PL) spectra of Compound 3 according to an embodiment of the present invention in a solution; and

FIG. 3 is a graph illustrating the voltage-efficiency characteristics of an organic light-emitting diode according to an embodiment of the present invention.

### DETAILED DESCRIPTION OF THE INVENTION

Embodiments of the present invention will now be described in more detail.

An aromatic compound according to an embodiment of the present invention is represented by Formula 1 below:

M₁-(B)ₙ-M₂ <Formula 1>

The aromatic compound of Formula 1 may be included in an organic layer interposed between a first electrode and a second electrode of an organic light-emitting diode. The aromatic compound of Formula 1 is suitable for use in a light-emitting layer, a hole injection layer, a hole transport layer, a hole blocking layer, or an electron transport layer of an organic light-emitting diode. The aromatic compound can be used both as a host material and a dopant material in a light-emitting layer.

In Formula 1, B is a single bond, a substituted or unsubstituted C₁-C₆₀ alkylene group, a substituted or unsubstituted C₅-C₆₀ cycloalkylene group, a substituted or unsubstituted C₅-C₆₀ heterocycloalkylene group, a substituted or unsubstituted C₅-C₆₀ arylene group, a substituted or unsubstituted C₂-C₆₀ heteroarylene group, or a divalent linking group represented by —N(Z₁)- where Z₁ is hydrogen, a substituted or unsubstituted C₁-C₆₀ alkyl group, or a substituted or unsubstituted C₅-C₆₀ aryl group.

Preferably, B may be a single bond, a substituted or unsubstituted C₁-C₁₀ alkylene group, a substituted or unsubstituted C₅-C₂₂ cycloalkylene group, a substituted or unsubstituted C₅-C₂₂ heterocycloalkylene group, a substituted or unsubstituted C₅-C₂₂ arylene group, a substituted or unsubstituted C₂-C₂₂ heteroarylene group, or a divalent linking group represented by —N(Z₁)- where Z₁ may be hydrogen, a substituted or unsubstituted C₁-C₁₀ alkyl group, or a substituted or unsubstituted C₅-C₂₂ aryl group. When B is a single bond, M₁ and M₂ may be directly connected.

More preferably, B may be a single bond, an ethylene group, a propylene group, a cyclohexylene group, a phenylene group, a naphthylene group, a phenalenylene group, an anthracenylene group, a fluorenylene group, a pyridinylene group, a thiophenylene group, or a divalent linking group represented by —N(Z₁)- where Z₁ may be a substituted or unsubstituted phenyl group, but is not limited thereto.

In Formula 1, n is an integer of 1 to 10. When n is 2 or more, two or more Bs may be the same or different. Preferably, n may be 1, 2, 3, 4, or 5, but is not limited thereto.

In more detail, in Formula 1, B may be a single bond, or —(B)ₙ- may be represented by one of structures of Formulas 3a through 3v below, but the present invention is not limited thereto:

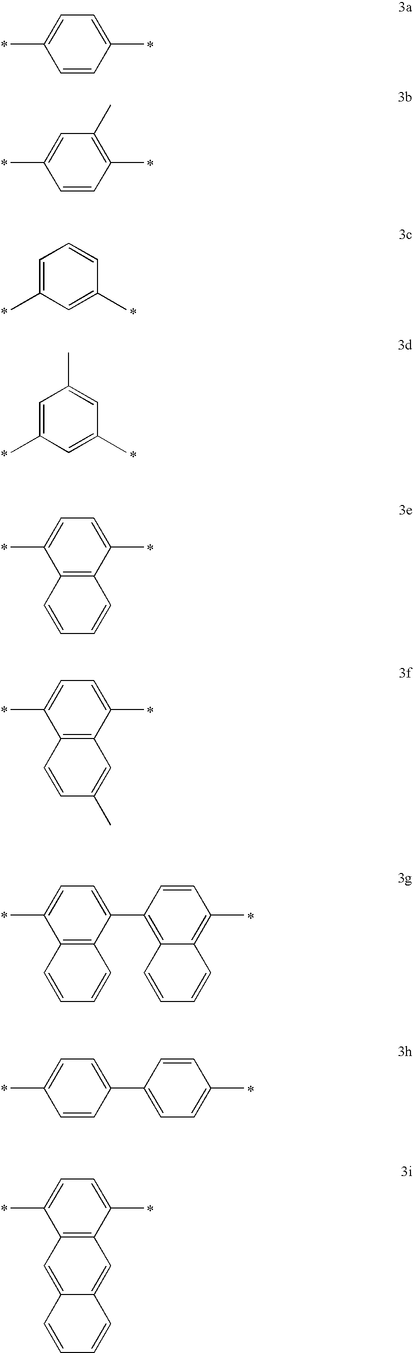

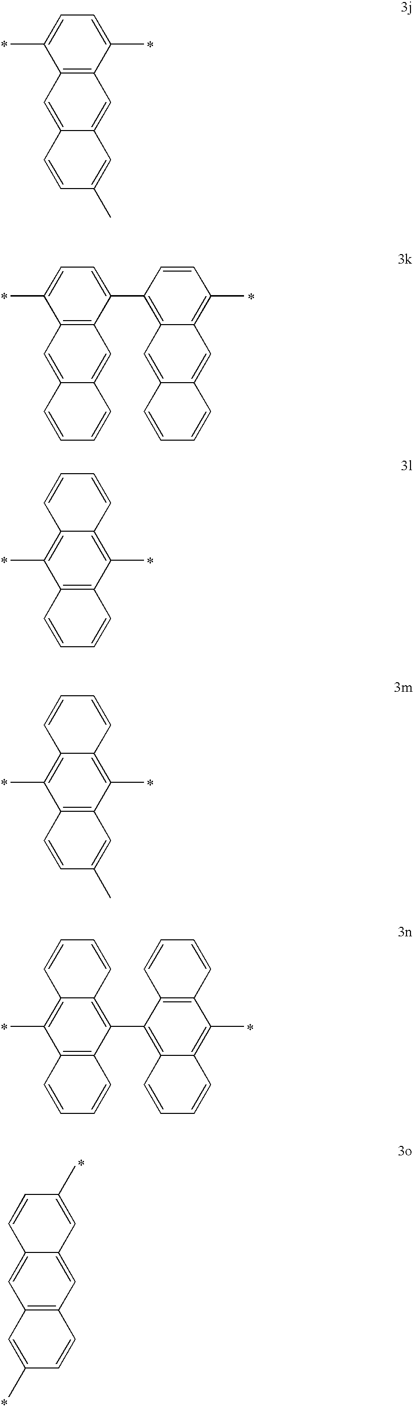

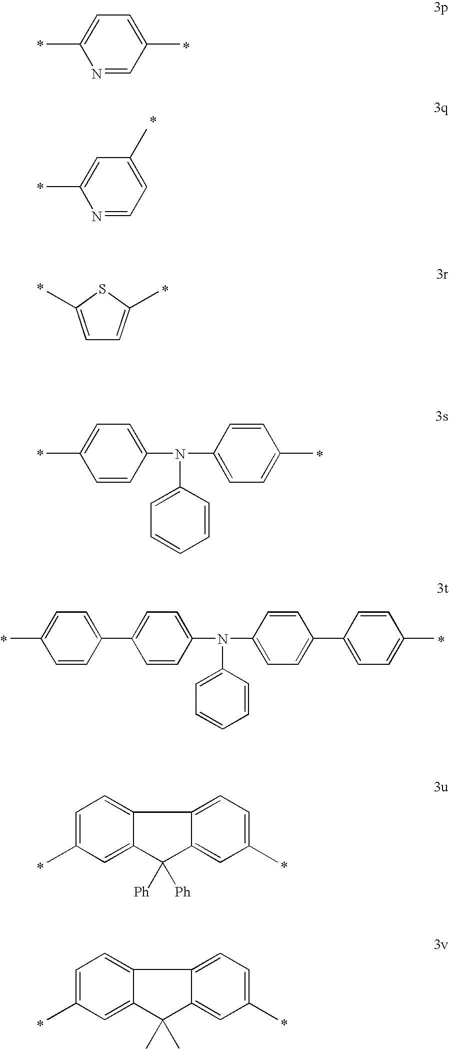

wherein two asterisks (*) of each structure respectively represent binding sites with M₁ and M₂ of Formula 1, and Ph represents a phenyl group.

M₁ and M₂ may be the same or different.

In Formula 1, M₁ and M₂ are each independently a terminal group derived from a compound represented by Formula 2 below:

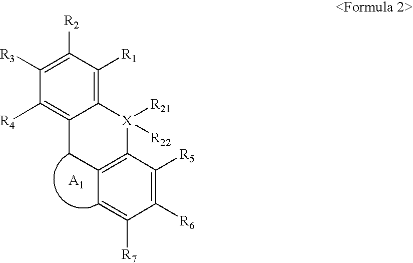

In Formula 2, R₂, and R₂₂ serve to increase solubility in a solvent and amorphous characteristics of the aromatic compound of Formula 1 to thereby enhance film processability.

Throughout the specification including the claims, the “terminal group derived from a compound represented by Formula 2” is a term used to describe that any atom forming rings represented by A₁, A₂, A₃, and A₄ in Formula 2′ below can be connected to B of Formula 1. This can be easily recognized by one of ordinary skill in the art by referring to terminal group structures represented by Formulas 4a through 4u and Compounds 1 through 26 represented by Formulas 5 through 30 as will be described later.

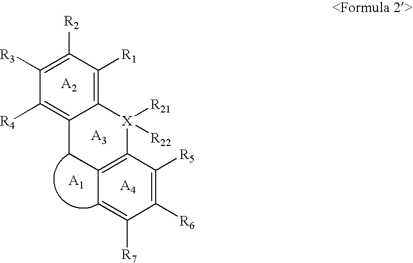

In Formula 2, X is a Group XIV element. Preferably, X may be C, Si, or Ge, but is not limited thereto.

In Formula 2, A₁ may be a substituted or unsubstituted benzene, a substituted or unsubstituted pentalene, a substituted or unsubstituted indene, a substituted or unsubstituted naphthalene, a substituted or unsubstituted azulene, a substituted or unsubstituted heptalene, a substituted or unsubstituted biphenylene, a substituted or unsubstituted indacene, a substituted or unsubstituted acenaphthylene, a substituted or unsubstituted fluorene, a substituted or unsubstituted phenalene, a substituted or unsubstituted phenanthrene, a substituted or unsubstituted anthracene, a substituted or unsubstituted fluoranthene, a substituted or unsubstituted acephenanthrylene, a substituted or unsubstituted aceanthrylene, a substituted or unsubstituted triphenylene, a substituted or unsubstituted pyrene, a substituted or unsubstituted chrysene, a substituted or unsubstituted naphthacene, a substituted or unsubstituted picene, a substituted or unsubstituted perylene, a substituted or unsubstituted pentaphene, a substituted or unsubstituted pentacene, a substituted or unsubstituted tetraphenylene, a substituted or unsubstituted hexaphene, a substituted or unsubstituted hexacene, a substituted or unsubstituted rubicene, a substituted or unsubstituted coronene, a substituted or unsubstituted pyranthrene, a substituted or unsubstituted ovalene, a substituted or unsubstituted thiophene, a substituted or unsubstituted indole, a substituted or unsubstituted furan, a substituted or unsubstituted benzothiophene, a substituted or unsubstituted parathiazine, a substituted or unsubstituted benzofuran, a substituted or unsubstituted pyrrole, a substituted or unsubstituted pyrazole, a substituted or unsubstituted imidazole, a substituted or unsubstituted imidazoline, a substituted or unsubstituted oxazole, a substituted or unsubstituted thiazole, a substituted or unsubstituted triazole, a substituted or unsubstituted tetrazole, a substituted or unsubstituted oxadiazole, a substituted or unsubstituted pyridine, a substituted or unsubstituted pyridazine, a substituted or unsubstituted pyrazine, a substituted or unsubstituted pyrimidine, a substituted or unsubstituted indole, a substituted or unsubstituted benzimidazole, a substituted or unsubstituted quinoline, a substituted or unsubstituted phenothiazine, or a substituted or unsubstituted thianthrene, but is not limited thereto. Of those, a substituted or unsubstituted benzene, a substituted or unsubstituted naphthalene, or a substituted or unsubstituted phenanthrene is preferred.

The naming of A₁ is done on the assumption that A₁ is separated from the compound of Formula 2. This can be easily recognized by one of ordinary skill in the art by referring to the terminal group structures of Formulas 4a through 4u and Compounds 1-26 represented by Formulas 5 through 30 as will be described later.

In Formula 2, R₁, R₂, R₃, R₄, R₅, R₆, R₇, R₂₁, and R₂₂ are each independently hydrogen, halogen, a cyano group, an amino group, a nitro group, a hydroxyl group, a substituted or unsubstituted C₁-C₆₀ alkyl group, a substituted or unsubstituted C₁-C₆₀ alkoxy group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₂-C₆₀ heteroaryl group, a substituted or unsubstituted C₅-C₆₀ arylamino group, a substituted or unsubstituted C₁-C₆₀ alkylamino group, a substituted or unsubstituted C₅-C₆₀ arylsilyl group, or a substituted or unsubstituted C₁-C₆₀ alkylsilyl group. Here, two or more of R₁, R₂, R₃, R₄, R₅, R₆, R₇, R₂₁, and R₂₂ may be optionally connected or fused together to form a substituted or unsubstituted C₆-C₆₀ aromatic ring, or a substituted or unsubstituted C₆-C₆₀ heteroaromatic ring.

Preferably, R₁, R₂, R₃, R₄, R₅, R₆, R₇, R₂₁, and R₂₂ may be each independently selected from the group consisting of hydrogen, a C₁-C₆₀ alkyl group, a C₂-C₆₀ alkenyl group, a C₂-C₆₀ alkynyl group, a C₅-C₆₀ cycloalkyl group, a C₅-C₆₀ cycloalkenyl group, a C₅-C₆₀ cycloalkynyl group, a cyclohexyl group, a phenyl group, a biphenyl group, a pentalenyl group, an indenyl group, a naphthyl group, a biphenylenyl group, an anthracenyl group, an azulenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxiranyl group, a pyrrolidinyl group, a pyrazolidinyl group, an imidazolidinyl group, a piperidinyl group, a piperazinyl group, a morpholinyl group, a di(C₅-C₆₀ aryl)amino group, a tri(C5-C60 alkyl)silyl group, a tri(C₅-C₆₀ aryl)silyl group, a diphenylaminophenyl group, a ditolylaminophenyl group, and derivatives thereof. As used herein, the term “derivative(s)” refers to the above-described group(s) wherein at least one hydrogen is substituted by the above-described substituent(s). For example, at least one hydrogen of the above-described group(s) may be substituted by a substituent selected from the group consisting of —F, —Cl, —Br, —CN, —NO₂, —NH₂, —OH, a C₁-C₆₀ alkyl group which is unsubstituted or substituted by a C₁-C₆₀ alkoxy group, —F, —Cl, —Br, —CN, —NO₂, —NH₂, or —OH, a C₅-C₆₀ cycloalkyl group which is unsubstituted or substituted by a C₁-C₆₀ alkyl group, a C₁-C₆₀ alkoxy group, —F, —Cl, —Br, —CN, —NO₂, —NH₂, or —OH, a C₅-C₆₀ aryl group which is unsubstituted or substituted by a C₁-C₆₀ alkyl group, a C₁-C₆₀ alkoxy group, —F, —Cl, —Br, —CN, —NO₂, —NH₂, or —OH, and a C₂-C₆₀ heteroaryl group which is unsubstituted or substituted by a C₁-C₆₀ alkyl group, a C₁-C₆₀ alkoxy group, —F, —Cl, —Br, —CN, —NO₂, —NH₂, or —OH.

Two or more of R₁, R₂, R₃, R₄, R₅, R₆, R₇, R₂₁, and R₂₂ may be optionally connected or fused together to form, for example, naphthalene or anthracene, but are not limited thereto.

More preferably, R₁, R₂, R₃, R₄, R₅, R₆, and R₇ may be each independently selected from the group consisting of hydrogen, a methyl group, a cyclohexyl group, a phenyl group, a biphenyl group, a tolyl group, a naphthyl group, a pyrenyl group, a phenanthrenyl group, a fluorenyl group, an imidazolinyl group, an indolyl group, a quinolinyl group, a diphenylamino group, a N,N-diphenylaminophenyl group, a N,N-di-p-tolylaminophenyl group, a trimethylsilyl group, a triphenylsilyl group, and derivatives thereof. R₂₁ and R₂₂ may be each independently hydrogen, —CH₃, —C₆H₁₁, or a phenyl group.

In Formula 2, at least one hydrogen of the alkylene group, the cycloalkylene group, the heterocycloalkylene group, the arylene group, the heteroarylene group, the alkyl group, the alkoxy group, the alkenyl group, the alkynyl group, the cycloalkyl group, the cycloalkenyl group, the aryl group, and the heteroaryl group may be substituted by a substituent selected from the group consisting of —F, —Cl, —Br, −CN, —NO₂, —NH₂, —OH, a C₁-C₆₀ alkyl group which is unsubstituted or substituted by a C₁-C₆₀ alkoxy group, —F, —Cl, —Br, —CN, —NO₂, —NH₂, or —OH, a C₅-C₆₀ cycloalkyl group which is unsubstituted or substituted by a C₁-C₆₀ alkyl group, a C₁-C₆₀ alkoxy group, —F, —Cl, —Br, —CN, —NO₂, —NH₂, or —OH, a C₅-C₆₀ aryl group which is unsubstituted or substituted by a C₁-C₆₀ alkyl group, a C₁-C₆₀ alkoxy group, —F, —Cl, —Br, —CN, —NO₂, —NH₂, or —OH, and a C₂-C₆₀ heteroaryl group which is unsubstituted or substituted by a C₁-C₆₀ alkyl group, a C₁-C₆₀ alkoxy group, —F, —Cl, —Br, —CN, —NO₂, —NH₂, or —OH.

In more detail, the compound of Formula 2 may be a compound represented by Formula 2a, 2b, or 2c below:

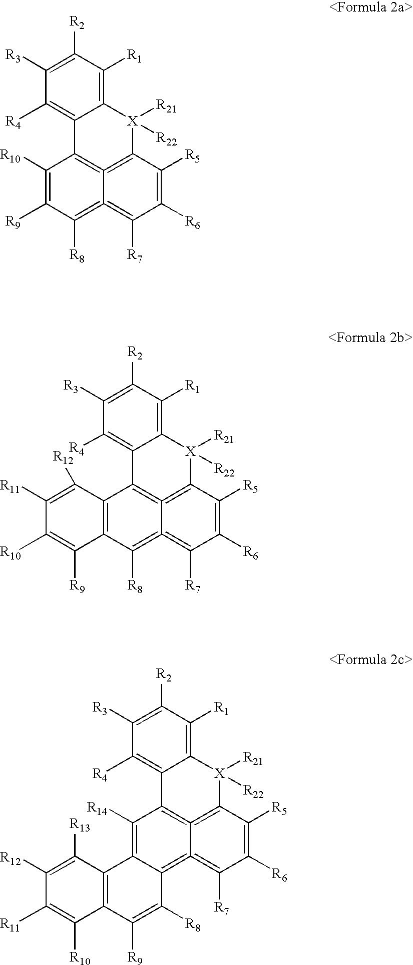

Formula 2a is an example of Formula 2 wherein A₁ is a substituted or unsubstituted benzene ring, Formula 2b is an example of Formula 2 wherein A₁ is a substituted or unsubstituted naphthalene ring, and Formula 2c is an example of Formula 2 wherein A₁ is a substituted or unsubstituted phenanthrene ring.

In Formulae 2a, 2b, and 2c, X, R₁, R₂, R₃, R₄, R₅, R₆, R₇, R₂₁, and R₂₂, and as defined in Formula 2. R₈, R₁, R₁₀, R₁₁, R₁₂, R₁₃, and R₁₄ in Formulae 2a, 2b, and 2c are as described above with reference to R₁, R₂, R₃, R₄, R₅, R₆, R₇, R₂₁, and R₂₂, and thus, a detailed description thereof will be omitted.

In more detail, M₁ and M₂, each of which is a terminal group derived from the compound of Formula 2, may be each independently represented by one of structures of Formulas 4a through 4u below, but are not limited thereto:

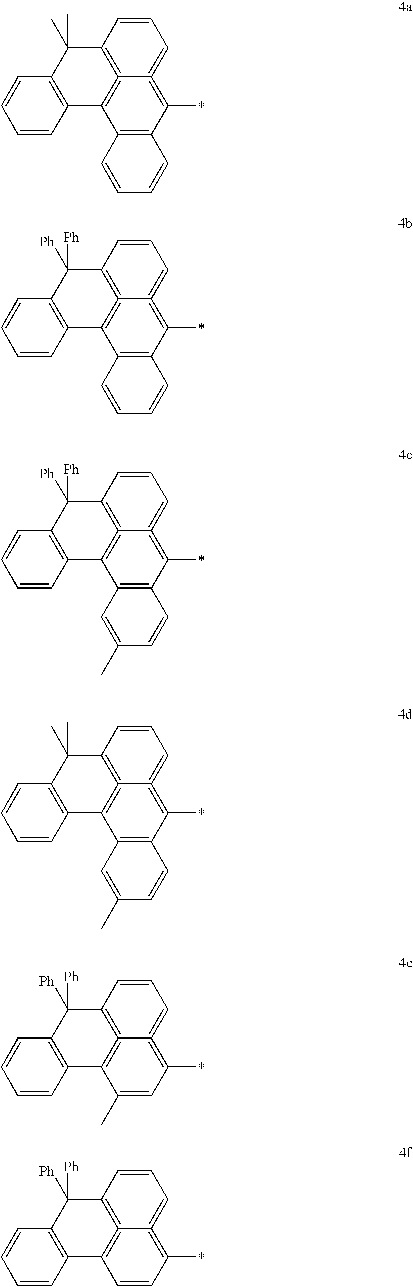

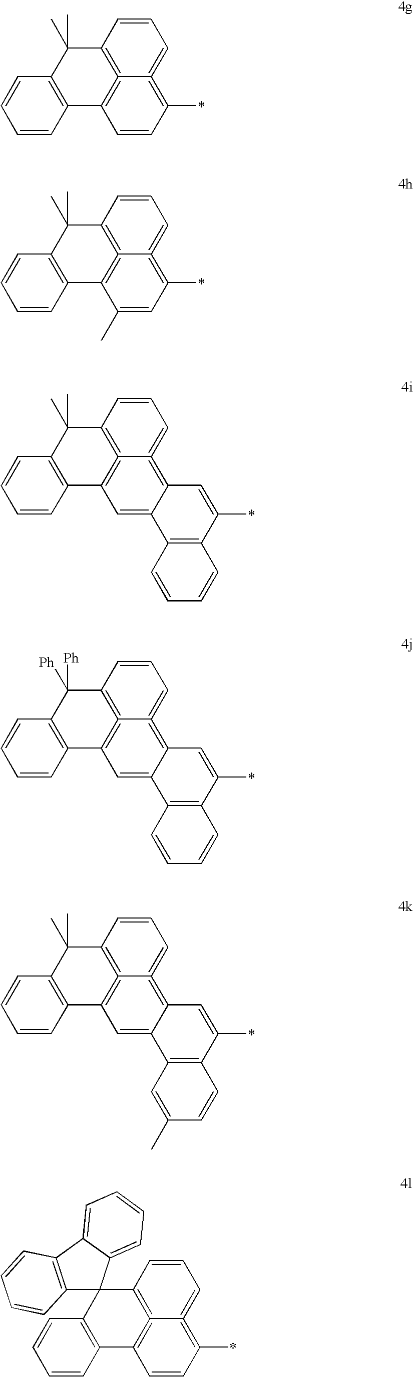

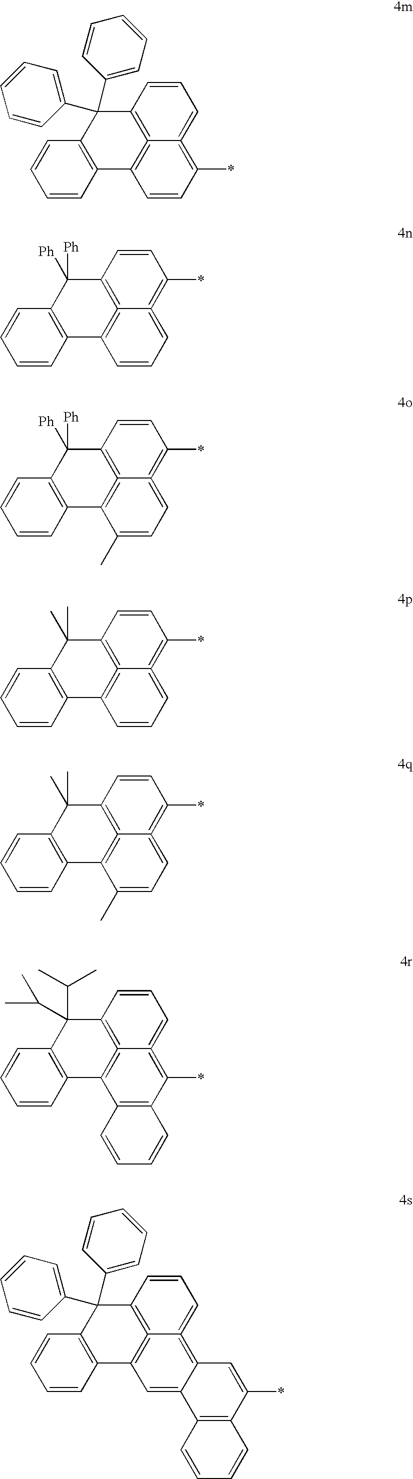

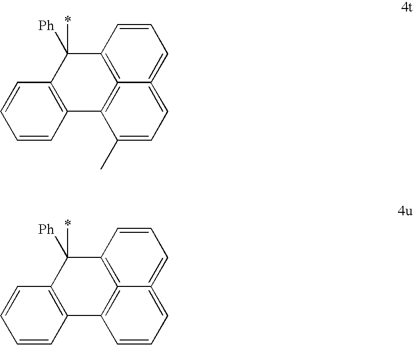

wherein an asterisk (*) of each structure represents a binding site with B of Formula 1, and Ph represents a phenyl group.

Specifically, the aromatic compound of Formula 1 according to embodiments of the present invention may be one of Compounds 1-26 represented by Formulas 5 through 30 below, but is not limited thereto:

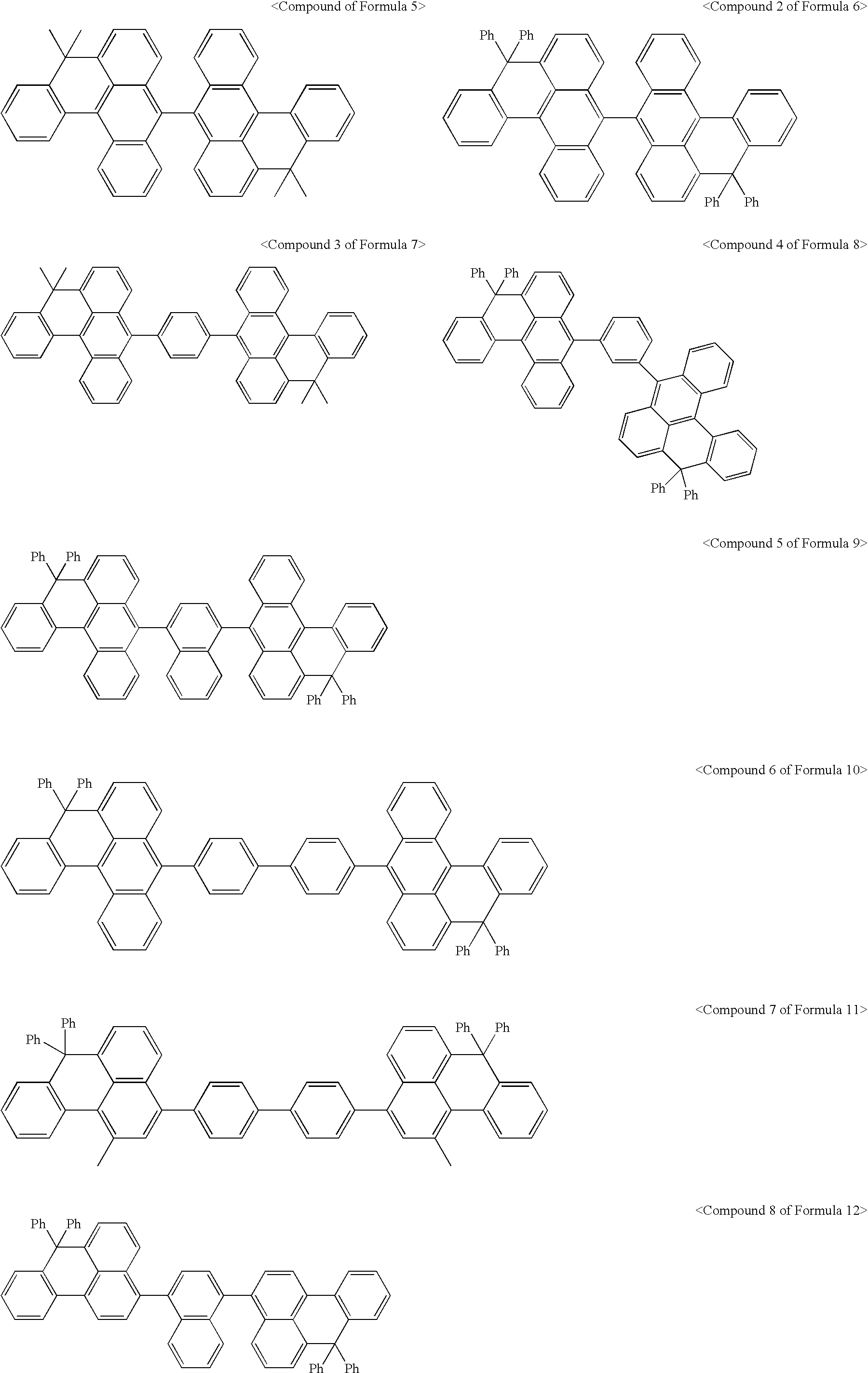

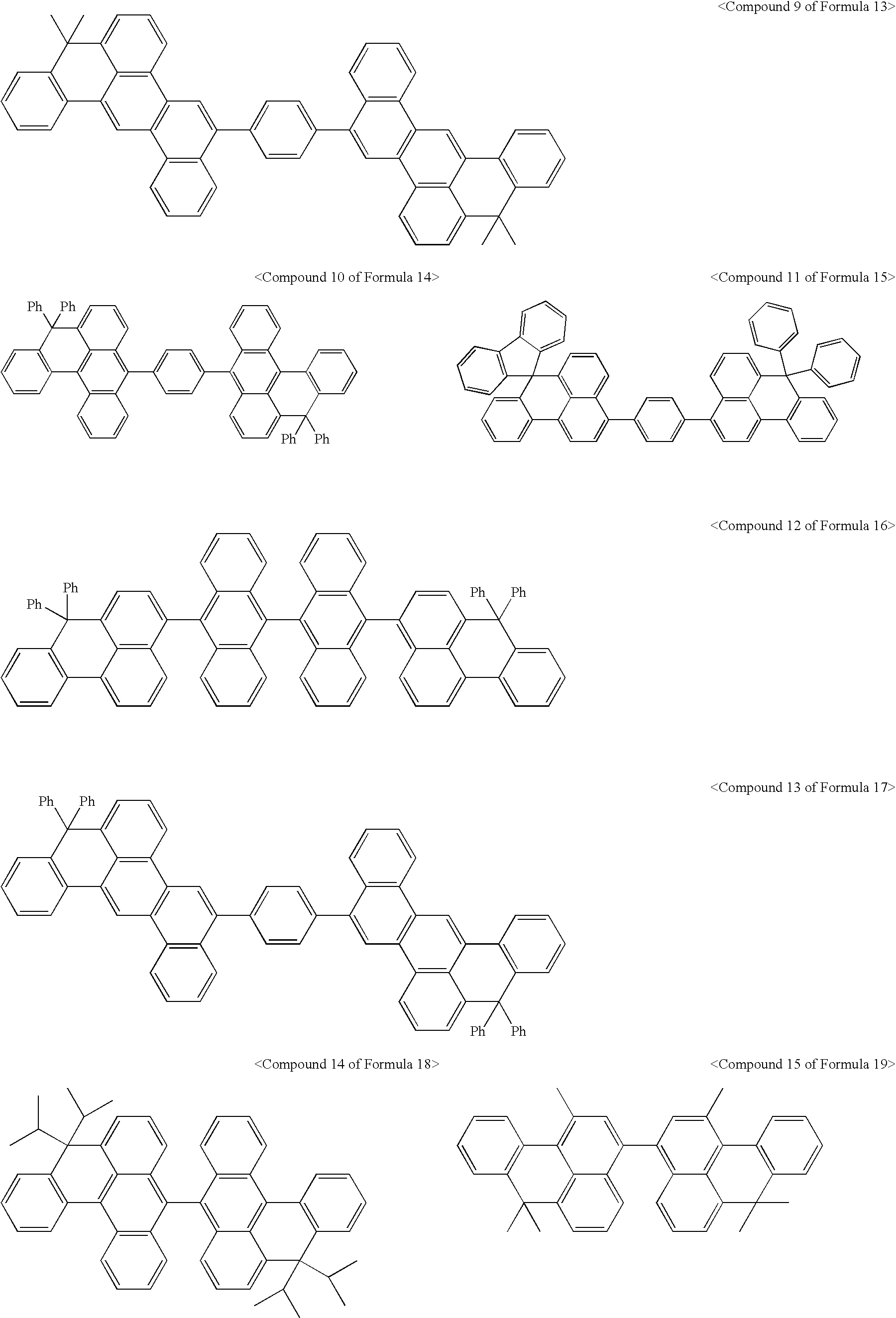

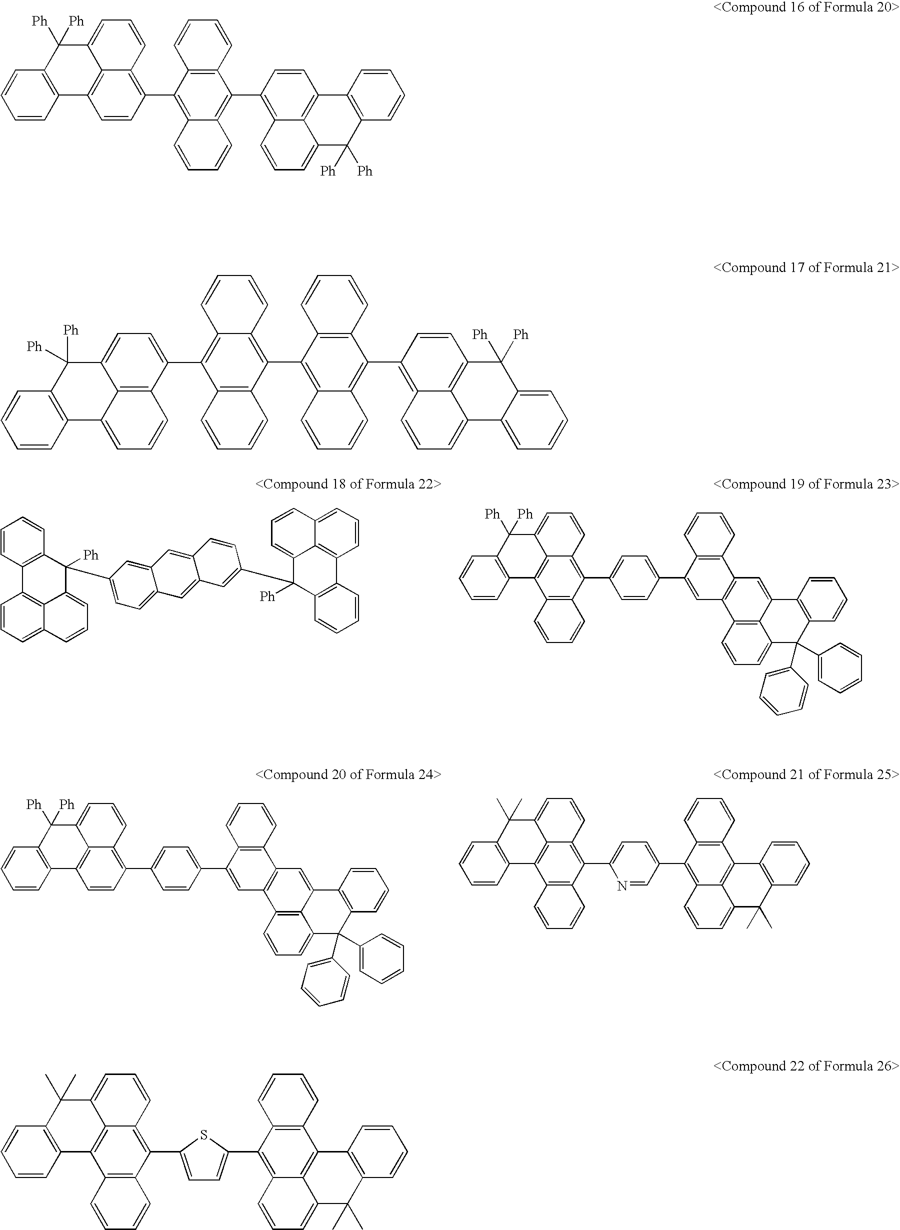

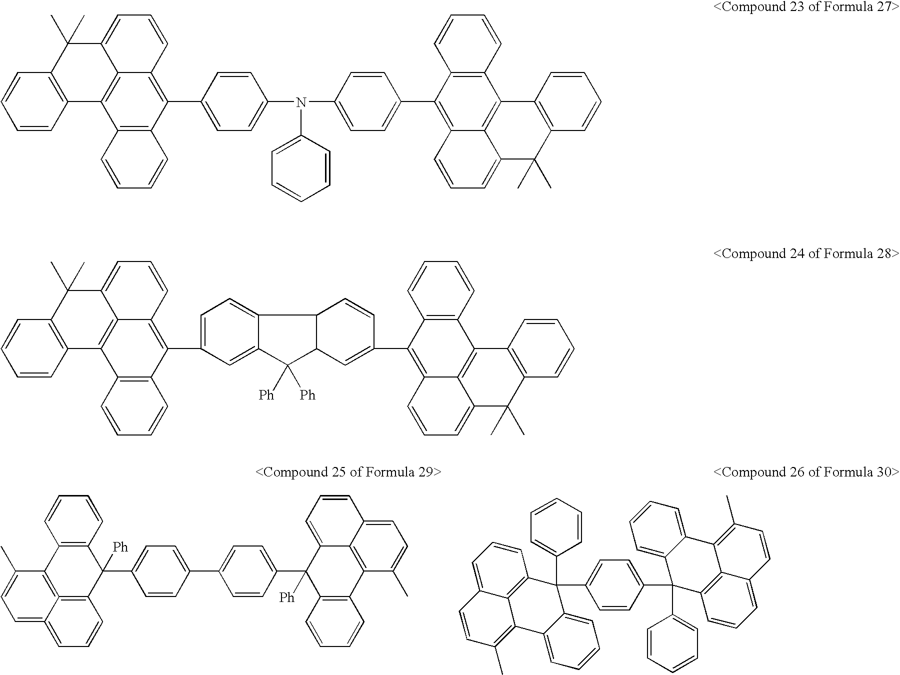

Throughout the specification, an unsubstituted aryl group refers to a monovalent group having an aromatic ring system and may contain two or more ring systems. Rings in the two or more ring systems may be optionally connected to each other or may be fused. An unsubstituted heteroaryl group refers to a group having a heteroaromatic ring system, that is, an aryl group in which at least one carbon atom of the ring(s) is substituted by at least one selected from the group consisting of N, O, S, and P. A cycloalkyl group refers to an alkyl group having a ring system, and a heterocycloalkyl group refers to a cycloalkyl group in which at least one carbon atom of the ring(s) is substituted by at least one selected from the group consisting of N, O, S, and P. An aromatic ring or a heteroaromatic ring is present in a fused form with a backbone of Formula 2, and may contain a single ring or two or more ring systems. Rings in the two or more ring systems may be connected to each other or may be fused. At least one hydrogen of the aromatic ring and the heteroaromatic ring may be substituted by substituent(s) as defined in R₈ through R₁₄.

The aromatic compound of Formula 1 according to an embodiment of the present invention can be synthesized using a conventional organic synthesis method. For detailed synthesis procedure for the aromatic compound, a reference may be made to the reaction schemes in the following synthesis examples.

Among compounds represented by Formula 2 according to an embodiment of the present invention, compounds in which X is Si or Ge can be synthesized according to Reaction Scheme 1a below:

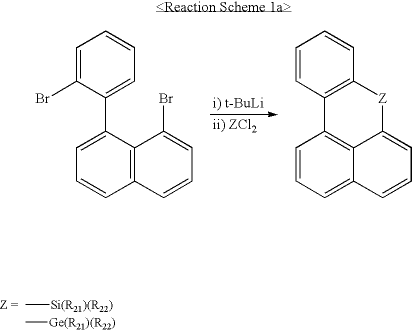

According to Reaction Scheme 1a, compounds in which X is Si or Ge can be obtained by replacing two bromo groups of 1-(2-bromophenyl)-8-bromonaphthalene with lithium and reacting the resultant products with ZCl₂. Compounds in which X is Si or Ge and R₁, R₂, R₃, R₄, R₆ and/or R₇ is not hydrogen can be easily synthesized by one of ordinary skill in the art by referring to Reaction Scheme 1a.

The above-described aromatic compound of Formula 1 can be included in an organic layer of an organic light-emitting diode. Thus, an organic light-emitting diode according to an embodiment of the present invention includes a first electrode, a second electrode, and an organic layer which is interposed between the first electrode and the second electrode and which includes the above-described aromatic compound of Formula 1.

Here, the organic layer may be a light-emitting layer, a hole injection layer, a hole transport layer, a hole blocking layer, or an electron transport layer.

The organic layer including the above-described aromatic compound of Formula 1 can be formed by various known methods. For example, a vacuum deposition method or a solution coating method such as spin coating, inkjet printing, screen printing, casting, Langmuir-Blodgett (LB) method, or spray printing may be used. A thermal transfer method may also be used by forming an organic layer including an aromatic compound of Formula 1 on a donor film using a vacuum deposition method or a solution coating method and thermally transferring the organic layer onto a substrate having thereon a first electrode. When using a solution coating method, a conventional organic light-emitting diode includes an organic layer with poor stability, but the organic light-emitting diode according to an embodiment of the present invention can have a low driving voltage, high efficiency, and high brightness since the aromatic compound of Formula 1 has excellent solubility and thermal stability and can form a stable organic layer.

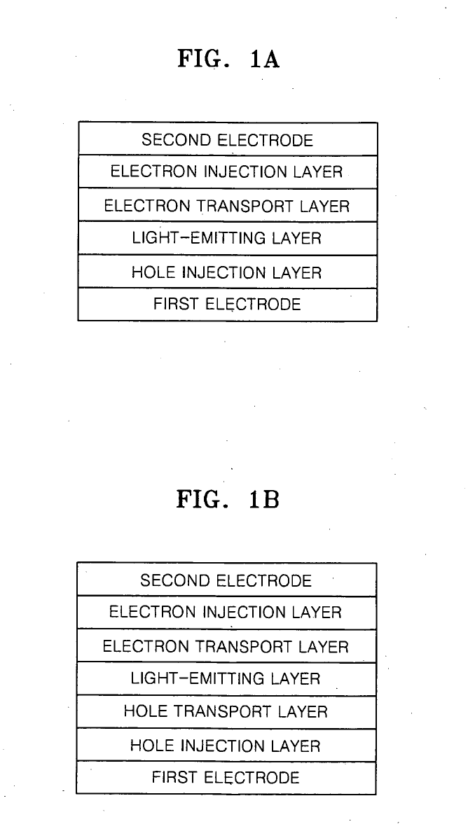

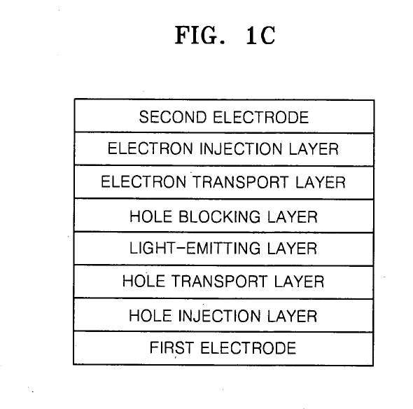

The organic light-emitting diode according to an embodiment of the present invention may further include at least one layer selected from the group consisting of a hole injection layer, a hole transport layer, a hole blocking layer, an electron transport layer, and an electron injection layer, between the first electrode and the second electrode. In more detail, organic light-emitting diodes according to embodiments of the present invention are illustrated in FIGS. 1A, 1B, and 1C. Referring to FIG. 1A, an organic light-emitting diode has a first electrode/hole injection layer/light-emitting layer/electron transport layer/electron injection layer/second electrode structure. Referring to FIG. 1B, an organic light-emitting diode has a first electrode/hole injection layer/hole transport layer/light-emitting layer/electron transport layer/electron injection layer/second electrode structure. Referring to FIG. 1C, an organic light-emitting diode has a first electrode/hole injection layer/hole transport layer/light-emitting layer/hole blocking layer/electron transport layer/electron injection layer/second electrode structure. Here, at least one of the light-emitting layer, the hole injection layer, the hole transport layer, the hole blocking layer, and the electron transport layer may include the above-described aromatic compound of Formula 1.

The present invention also provides a method of manufacturing an organic light-emitting diode.

In the method, a first electrode is formed, and an organic layer including a compound of Formula 1 as described above is formed on the first electrode. Then, a second electrode is formed on the organic layer.

Here, the organic layer may be a light-emitting layer, a hole injection layer, a hole transport layer, a hole blocking layer, or an electron transport layer. If necessary, the method may further include forming at least one layer selected from the group consisting of a hole injection layer, a hole transport layer, a light-emitting layer, a hole blocking layer, an electron transport layer, and an electron injection layer.

The formation of the organic layer including the aromatic compound of Formula 1 may be performed using a vacuum deposition method or a solution coating method such as spin coating, inkjet printing, screen printing, casting, LB method, or spray printing. A thermal transfer method may also be performed by forming an organic layer including an aromatic compound of Formula 1 on a donor film using a vacuum deposition process or a solution coating process and thermally transferring the organic layer onto a substrate having thereon a first electrode.

Hereinafter, a method of manufacturing an exemplary organic light-emitting diode according to an embodiment of the present invention will be described with reference to FIG. 1C.

First, a first electrode material with a high work function is applied onto a substrate using deposition or sputtering to form a first electrode. The first electrode may be an anode. Here, the substrate may be a substrate commonly used in organic light-emitting diodes. Preferably, the substrate may be a glass or transparent plastic substrate which has excellent mechanical strength, thermal stability, transparency, surface smoothness, handling properties, and water repellency. The first electrode material may be a material with transparency and good conductivity, e.g., indium tin oxide (ITO), indium zinc oxide (IZO), tin oxide (SnO₂), or zinc oxide (ZnO).

Next, a hole injection layer (HIL) may be formed on the first electrode using various methods such as vacuum deposition, spin-coating, casting, or LB method.

When forming the hole injection layer using a vacuum deposition process, the deposition conditions vary according to the type of a hole injection layer material, the structure and thermal characteristics of the hole injection layer, etc. However, it is preferred that the hole injection layer is deposited at a deposition rate of 0.01 to 100 Å/sec, at a temperature of 100 to 500° C., in a vacuum level of 10⁻⁸ to 10⁻³ torr.

When forming the hole injection layer using a spin-coating process, the coating conditions vary according to the type of a hole injection layer material, the structure and thermal characteristics of the hole injection layer, etc. However, it is preferred that the spin-coating is performed at a coating speed of about 2000 to 5000 rpm, and, after the spin-coating, a thermal treatment is performed at a temperature of about 80 to 200° C. for the purpose of solvent removal.

The hole injection layer material may be an aromatic compound of Formula 1 as described above. The hole injection layer material may also be a known hole injection material, e.g., a phthalocyanine compound (e.g., copper phthalocyanine) disclosed in U.S. Pat. No. 4,356,429 which is incorporated herein by reference, a Starburst-type amine derivative (e.g., TCTA, m-MTDATA, or m-MTDAPB) disclosed in Advanced Material, 6, p. 677 (1994) which is incorporated herein by reference, or a soluble conductive polymer, e.g., polyaniline/dodecylbenzenesulfonic acid (Pani/DBSA), poly(3,4-ethylenedioxythiophene)/poly(4-styrenesulfonate) (PEDOT/PSS), polyaniline/camphor sulfonic acid (Pani/CSA), or polyaniline/poly(4-styrenesulfonate) (PANI/PSS):

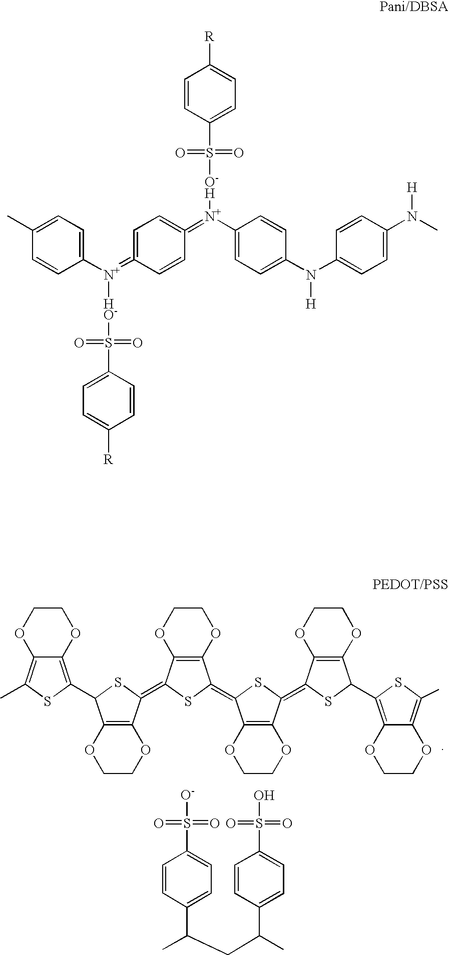

The thickness of the hole injection layer may be about 100 to 10,000 Å, preferably 100 to 1,000 Å. When the thickness of the hole injection layer satisfies the above range, satisfactory hole injection characteristics can be achieved with no substantial drop in driving voltage.

Next, a hole transport layer (HTL) may be formed on the hole injection layer using various methods such as vacuum deposition, spin-coating, casting, or LB method. When forming the hole transport layer using vacuum deposition or spin-coating, the deposition or coating conditions vary according to the type of the compound used, but are generally almost the same as those recited in the formation of the hole injection layer.

A hole transport layer material may be an aromatic compound of Formula 1 as described above. The hole transport layer material may also be a known hole transport material, e.g., a carbazole derivative such as N-phenylcarbazole or polyvinylcarbazole, a common amine derivative having an aromatic fused ring such as N,N′-bis(3-methylphenyl)-N,N′-diphenyl-[1,1-biphenyl]-4,4′-diamine (TPD) or N,N′-di(naphthalene-1-yl)-N,N′-diphenylbenzidine (α-NPD), etc.

The thickness of the hole transport layer may be about 50 to 1,000 Å, preferably 100 to 800 Å. When the thickness of the hole transport layer satisfies the above range, satisfactory hole transport characteristics can be achieved with no substantial drop in driving voltage.

Next, a light-emitting layer (EML) may be formed on the hole transport layer using vacuum deposition, spin-coating, casting, or LB method. When forming the light-emitting layer using vacuum deposition or spin-coating, the deposition or coating conditions vary according to the type of the compound used, but are generally almost the same as those recited in the formation of the hole injection layer.

The light-emitting layer may include an aromatic compound of Formula 1 as described above. Here, the aromatic compound of Formula 1 may be used as a dopant in combination with a suitable known host material. A known dopant material may be further included. The aromatic compound of Formula 1 may also be used alone. For example, the host material may be tris(8-quinolinolate)aluminum (Alq3), 4,4′-N,N′-dicarbazole-biphenyl (CBP), poly(n-vinylcarbazole) (PVK), 9,10-di(naphthalene-2-yl)anthracene (ADN) or the like, but is not limited thereto:

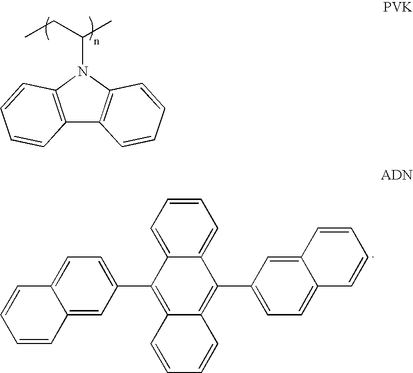

A known red dopant may be platinum octaethylporphyrin (PtOEP), Tris(1-phenylquinoline) iridium (III) Ir(piq)₃, bis[2-(2f-benzothienyl)pyridinato-N,C3 f] (acetylacetonato) iridium(II) (Btp₂Ir(acac)), 4-(dicyanomethylene)-2-t-butyl-6(1,1,7,7-tetramethyljulolidyl-9-enyl)-4H-pyran (DCJTB), or the like, but is not limited thereto:

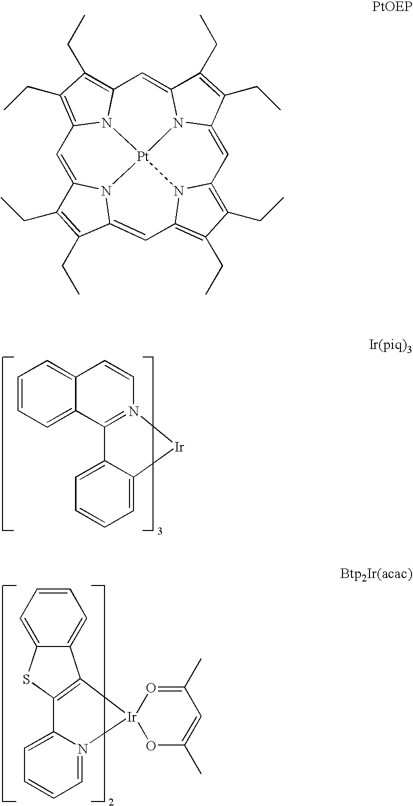

A known green dopant may be Ir(ppy)₃ (ppy=phenylpyridine), Ir(ppy)₂(acac), Ir(mpyp)₃, C545T, or the like, but is not limited thereto:

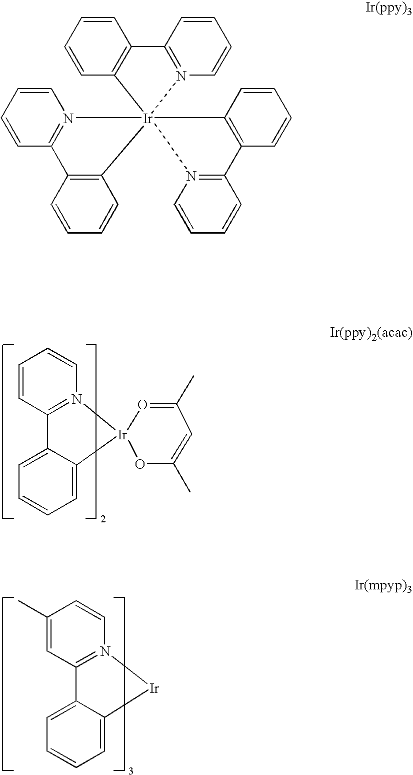

A known blue dopant may be F₂Irpic, (F₂ppy)₂Ir(tmd), Ir(dfppz)₃, ter-fluorene, or the like, but is not limited thereto:

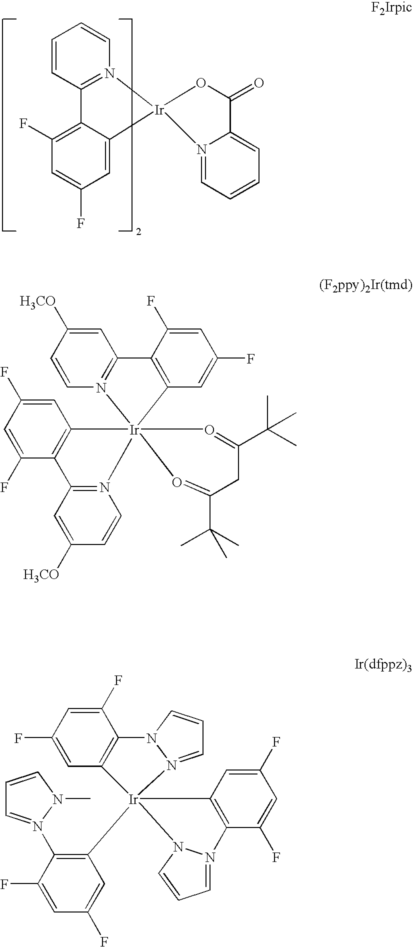

When using both a dopant and a host, the doping concentration of the dopant is not particularly limited. Generally, the content of the dopant is 0.01 to 15 parts by weight based on 100 parts by weight of the host.

The thickness of the light-emitting layer may be about 100 to 1,000 Å, preferably 200 to 600 Å. When the thickness of the light-emitting layer satisfies the above range, excellent emission characteristics can be achieved with no substantial drop in driving voltage.

When the light-emitting layer includes a phosphorescent dopant, a hole blocking layer (HBL) may be formed between the hole transport layer and the light-emitting layer using vacuum deposition, spin-coating, casting, or LB method, in order to prevent the diffusion of triplet excitons or holes into an electron transport layer. When forming the hole blocking layer using vacuum deposition or spin coating, the deposition or coating conditions vary according to the type of the compound used, but are generally almost the same as those recited in the formation of the hole injection layer. An available hole blocking material may be an oxadiazole derivative, a triazole derivative, a phenanthroline derivative, BCP, or a hole blocking material disclosed in Japanese Patent Laid-Open Publication No. Hei. 11-329734, etc.

The thickness of the hole blocking layer may be about 50 to 1,000 Å, preferably 100 to 300 Å. When the thickness of the hole blocking layer satisfies the above range, excellent hole blocking characteristics can be achieved with no substantial drop in driving voltage.

Next, an electron transport layer (ETL) may be formed using various methods such as vacuum deposition, spin-coating, or casting. When forming the electron transport layer using vacuum deposition or spin-coating, the deposition or coating conditions vary according to the type of the compound used, but are generally almost the same as those recited in the formation of the hole injection layer. An electron transport layer material serves to stably transport electrons from an electron donor electrode (a cathode) and may be an aromatic compound of Formula 1 as described above. A known electron transport material such as a quinoline derivative, in particular, aluminum(III) tris(8-hydroxyquinolate) (Alq3), 3-phenyl-4-(1′-naphthyl)-5-phenyl-1,2,4-triazole (TAZ), or aluminum(III)bis(2-methyl-8-quinolinato)4-phenylphenolate (Balq) may also be used, but the present invention is not limited thereto:

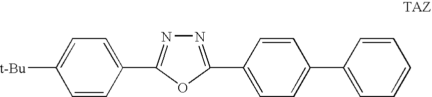

The thickness of the electron transport layer may be about 150 to 1,000 Å, preferably 200 to 500 Å. When the thickness of the electron transport layer satisfies the above range, satisfactory electron transport characteristics can be achieved with no substantial drop in driving voltage.

An electron injection layer (EIL) may be formed on the electron transport layer in order to facilitate the injection of electrons from a cathode. An electron injection layer material is not particularly limited.

The electron injection layer material may be optionally selected from known materials such as LiF, NaCl, CsF, Li₂O, or BaO. The deposition conditions of the electron injection layer vary according to the type of the compound used, but are generally almost the same as those recited in the formation of the hole injection layer.

The thickness of the electron injection layer may be about 1 to 100 Å, preferably 5 to 50 Å. When the thickness of the electron injection layer satisfies the above range, satisfactory electron injection characteristics can be achieved with no substantial drop in driving voltage.

Then, a second electrode may be formed on the electron injection layer using vacuum deposition or sputtering. The second electrode may be used as a cathode. A material for forming the second electrode may be metal or alloy with a low work function, an electroconductive compound, or a mixture thereof. For example, the second electrode material may be lithium (Li), magnesium (Mg), aluminum (Al), aluminum-lithium (Al—Li), calcium (Ca), magnesium-indium (Mg—In), magnesium-silver (Mg—Ag), etc. The second electrode may also be a transmissive cathode formed of ITO or IZO to provide a top emission type diode.

Hereinafter, the present invention will be described more specifically with reference to the following examples. However, the following examples are only for illustrative purposes and are not intended to limit the scope of the invention.

### EXAMPLES

### Synthesis Example 1

Compound 3 was synthesized according to Reaction Schemes 1-1 to 1-3 below.

### Synthesis of Intermediate B

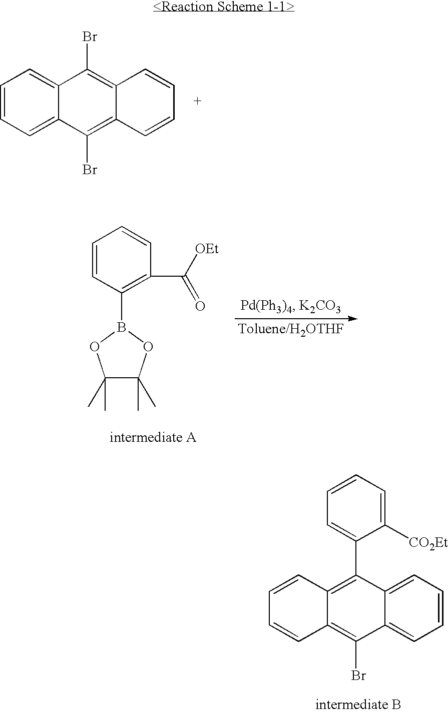

9.1 g (27.2 mmol) of 9,10-dibromoanthracene was dissolved in tetrahydrofuran (THF) (110 ml). Then, a solution of 7.5 g (27.2 mmol) of an intermediate A, 1.57 g (1.4 mmol) of tetrakis triphenylphosphine palladium (Pd(PPh₃)₄), and 1.5 g (109 mmol) of potassium carbonate (K₂CO₃) in 55 ml of toluene and 55 ml of water was added thereto, and the reaction mixture was refluxed for 24 hours. After the reaction was terminated, a solvent was removed by evaporation. The residue was washed with 500 ml of ethylacetate and 500 ml of water. The organic layer was collected and dried over anhydrous magnesium sulfate. The crude product was purified by silica chromatography to give 2.9 g (yield: 26%) of an intermediate B.

### Synthesis of Intermediate C

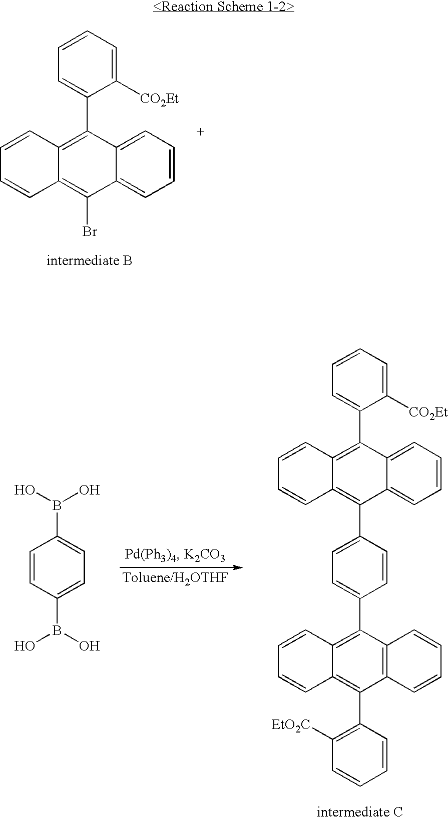

2.0 g (4.9 mmol) of the intermediate B was dissolved in THF (48 ml). Then, a solution of 409 g (2.5 mmol) of 4,4′-biphenyldiboronic acid, 290 mg (0.25 mmol) of tetrakis triphenylphosphine palladium (Pd(PPh₃)₄), and 3.4 mg (24.7 mmol) of potassium carbonate (K₂CO₃) in 12 ml of toluene and 12.5 ml of water was added thereto, and the reaction mixture was refluxed for 24 hours. After the reaction was terminated, a solvent was removed by evaporation. The residue was washed with 200 ml of ethylacetate and 200 ml of water. The organic layer was collected and dried over anhydrous magnesium sulfate. The crude product was purified by silica chromatography to give 1.4 g (yield: 77%) of an intermediate C.

### Synthesis of Compound 3

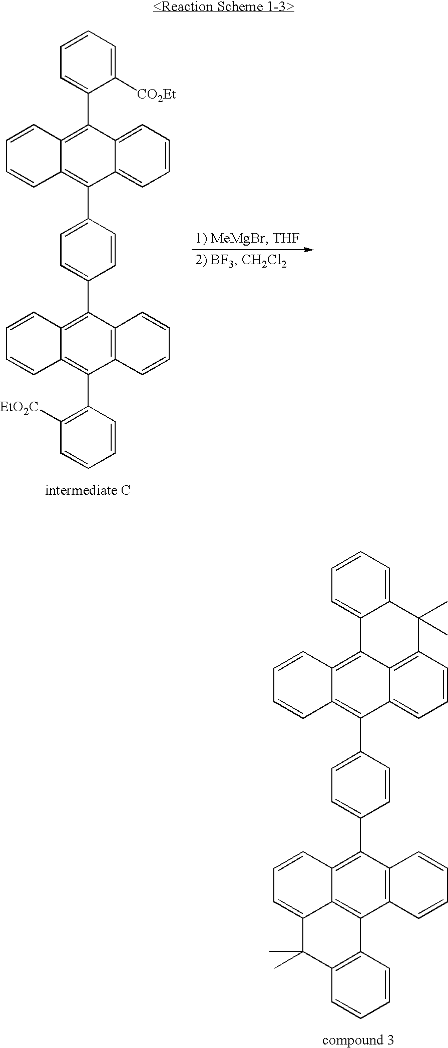

129 mg (0.2 mmol) of the intermediate C was dissolved in THF (4 ml), and methyl magnesium bromide (MeMgBr 3.0 M, 0.3 ml) was added thereto. The reaction mixture was heated to 70° C. and stirred for one hour. After the reaction was terminated, the reaction solution was washed with 10 ml of water and 10 ml of ethylacetate. The organic layer was collected, dried over anhydrous magnesium sulfate, and then dried under a reduced pressure. The resultant solid was dissolved in 4 ml of methylene chloride, and 0.2 ml of boron trifluoride-diethyl etherate was added thereto. The reaction mixture was stirred for 30 minutes. The reaction was terminated by adding 1 ml of methanol, and the reaction solution was then washed with 10 ml of methylene chloride and 10 ml of water. The organic layer was collected and dried over anhydrous magnesium sulfate. The crude product was purified by silica chromatography to give 100 mg (yield: 75%) of Compound 3.

¹H-NMR (CDCl₃, 300 MHz, ppm): 8.8 (d, 2H), 8.1-7.3 (m, 24H), 1.7 (s, 12H)

### Synthesis Example 2

Compound 10 was synthesized according to Reaction Scheme 2 below.

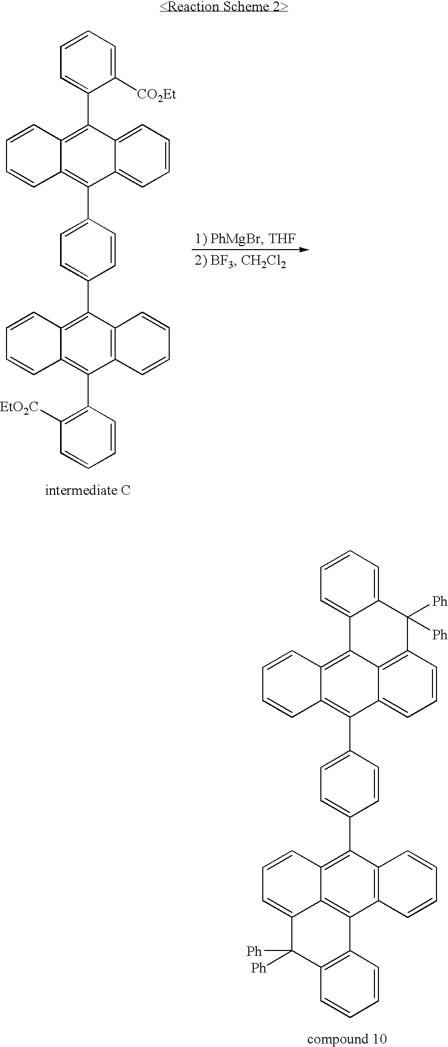

351 mg (yield: 70%) of Compound 10 was synthesized in the same manner as that of the synthesis of Compound 3 of Synthesis Example 1 except that phenyl magnesium bromide (PhMgBr) was used instead of methyl magnesium bromide (MeMgBr).

¹H-NMR (CDCl₃, 300 MHz, ppm): 8.8 (m, 2H), 8.2-6.9 (m, 44H)

### Synthesis Example 3

Compound 13 was synthesized according to Reaction Schemes 3-1 to 3-3 below:

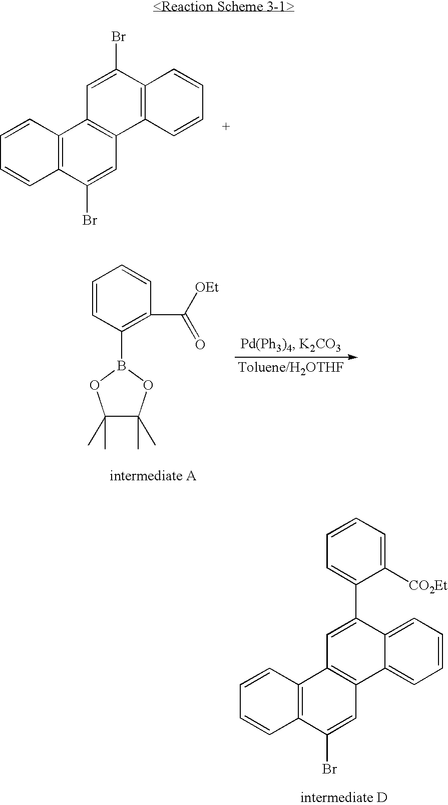

530 mg (yield: 90%) of an intermediate D was synthesized in the same manner as that of the synthesis of the intermediate B of Synthesis Example 1 except that 6,12-dibromochrysene was used instead of 9,10-dibromoanthracene.

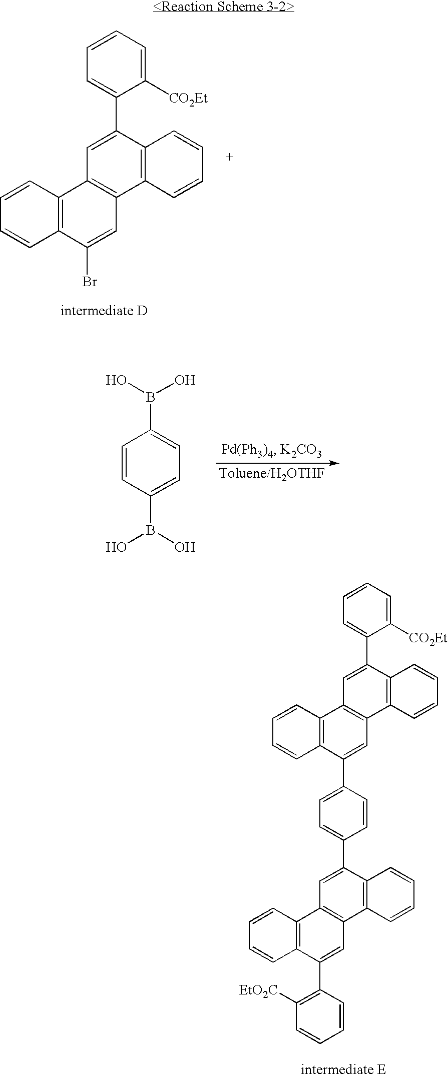

565 mg (yield: 72%) of an intermediate E was synthesized in the same manner as that of the synthesis of the intermediate C of Synthesis Example 1 except that the intermediate D was used instead of the intermediate B.

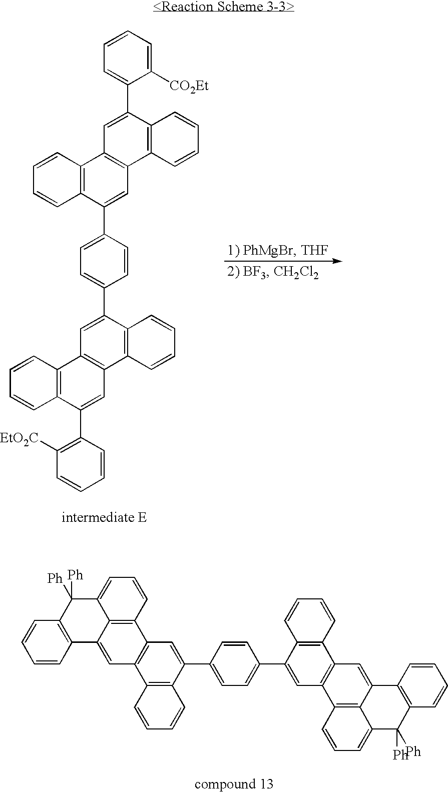

300 mg (yield: 20%) of Compound 13 was synthesized in the same manner as that of synthesis of Compound 3 of Synthesis Example 1 except that the intermediate E and phenyl magnesium bromide (PhMgBr) were used instead of the intermediate C and the methyl magnesium bromide (MeMgBr), respectively.

¹H-NMR (CDCl₃, 300 MHz, ppm): 9.3-6.9 (m, 50H)

### Synthesis Example 4

Compound 26 was synthesized according to Reaction Schemes 4-1 and 4-2 below:

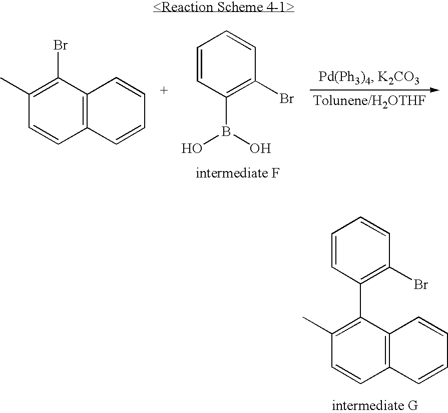

1.0 g (yield: 33%) of an intermediate G was synthesized in the same manner as that of the synthesis of the intermediate B of Synthesis Example 1 except that 1-bromo-2-methylnaphthalene and an intermediate F were used instead of the 9,10-dibromoanthracene and the intermediate A, respectively.

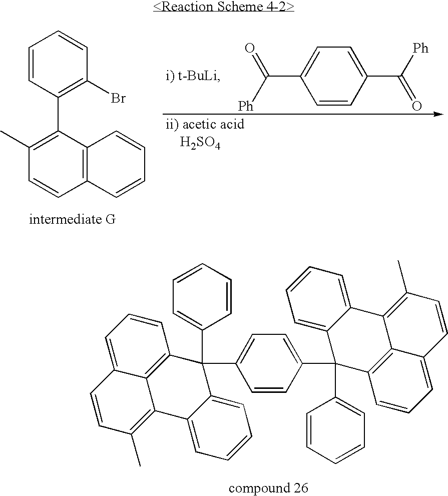

505 mg (1.7 mmol) of the intermediate G and tert-butyl lithium (t-BuLi 1.7 M, 1 ml) were added to 5 ml of TFT that had been cooled to −78° C. The reaction mixture was stirred for 30 minutes, and 300 mg (0.8 mmol) of 1,4-dibenzoylbenzene was added thereto. After the reaction was terminated, the reaction solution was washed with 50 ml of a 1 M HCl solution and 50 ml of ethylacetate. The organic layer was collected, dried over anhydrous magnesium sulfate, and then dried under a reduced pressure. The resultant solid was dissolved in 4 ml of an acetic acid, and 0.1 ml of a sulfuric acid was added thereto. The reaction mixture was stirred at 100° C. for three hours. After the reaction was terminated, a solid was filtered through a filter paper and washed with ethanol. The crude product was purified by silica chromatography to give 460 mg (yield: 40%) of Compound 26.

¹H-NMR (CDCl₃, 300 MHz, ppm): 8.5-6.9 (m, 32H), 2.2 (s, 6H)

### Evaluation Example 1

### Evaluation of Emission Characteristics of Compounds (in Solution Phase)

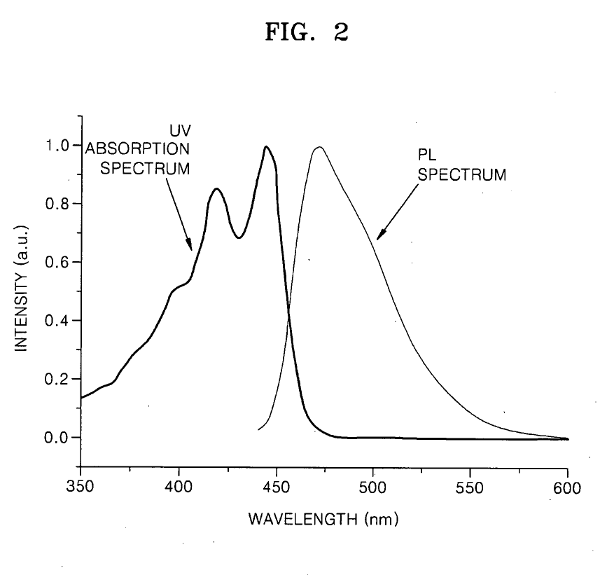

Emission characteristics of Compounds 3, 13, 10, and 26 were evaluated by measuring the UV absorption and PL (photoluminescence) spectra of Compounds 3, 13, 10, and 26. First, Compound 3 was diluted with toluene to a concentration of 0.2 mM, and UV absorption spectrum of the diluted solution was measured using Shimadzu UV-350 spectrometer. The same experiment was performed for Compounds 13, 10, and 26. Meanwhile, Compound 3 was diluted with toluene to a concentration of 10 mM, and PL spectrum of the diluted solution was measured using an ISC PC1 spectrofluorometer equipped with a Xenon lamp. The same experiment was performed for Compounds 13, 10, and 26. The results are presented in Table 1 below. In particular, the UV absorption and PL spectra of Compound 3 are shown in FIG. 2.

TABLE 1

| Compound | UV absorption wavelength (nm) | PL wavelength (nm) |
|---|---|---|
| 3 | 440 | 470 |
| 10 | 410 | 440 |
| 13 | 440 | 470 |
| 26 | 360 | 400 |

The above results show that the compounds according to the embodiments of the present invention has emission characteristics suitable for use in organic light-emitting diodes.

### Example 1

Organic light-emitting diodes having the following structure were manufactured using Compound 3 as a dopant of a light-emitting layer and ADN as a host of the light-emitting layer: ITO/α-NPD(750 Å)/Compound 3(5 wt %)+ADN(350 Å)/Alq3(180 Å)/LiF(10 Å)/Al(2000 Å).

Each organic light-emitting diode was formed as follows. A 15 Ω/cm² (1,000 Å) ITO glass substrate was cut into pieces of 50 mm×50 mm×0.7 mm in size, followed by ultrasonic cleaning in acetone, isopropyl alcohol, and pure water (for 15 minutes each) and then UV/ozone cleaning (for 30 minutes) to form an anode. Then, α-NPD was vacuum-deposited to a thickness of 750 Å on the ITO anode at a deposition rate of 1 Å/sec to form a hole transport layer. Then, Compound 3 and ADN were vacuum-deposited to a total thickness of 350 Å on the hole transport layer at a deposition rate of 5 Å/sec and 30 Å/sec, respectively, to form a light-emitting layer. Then, Alq3 was vacuum-deposited to a thickness of 180 Å on the light-emitting layer to form an electron transport layer. LiF (10 Å, electron injection layer) and Al (2,000 Å, cathode) were sequentially vacuum-deposited on the electron transport layer to thereby complete organic light-emitting diode as illustrated in FIG. 1A. The organic light-emitting diodes were designated as “sample 1”.

### Example 2

Organic light-emitting diodes were manufactured in the same manner as in Example 1 except that Compound 10 was used instead of Compound 3, and were designated as “sample 2”.

### Example 3

Organic light-emitting diodes were manufactured in the same manner as in Example 1 except that Compound 13 was used instead of Compound 3, and were designated as “sample 3”.

### Example 4

Organic light-emitting diodes were manufactured in the same manner as in Example 1 except that Compound 26 was used instead of Compound 3, and were designated as “sample 4”.

### Comparative Example 1

Organic light-emitting diodes were manufactured in the same manner as in Example 1 except that Compound 27 by Formula 31 was used instead of Compound 3, and were designated as “sample A”.

<Compound 27 of Formula 31>

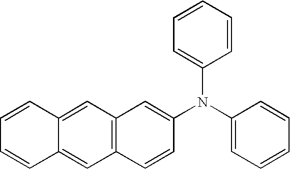

### Evaluation Example 2

### Evaluation of Characteristics of Samples 1-4 and A

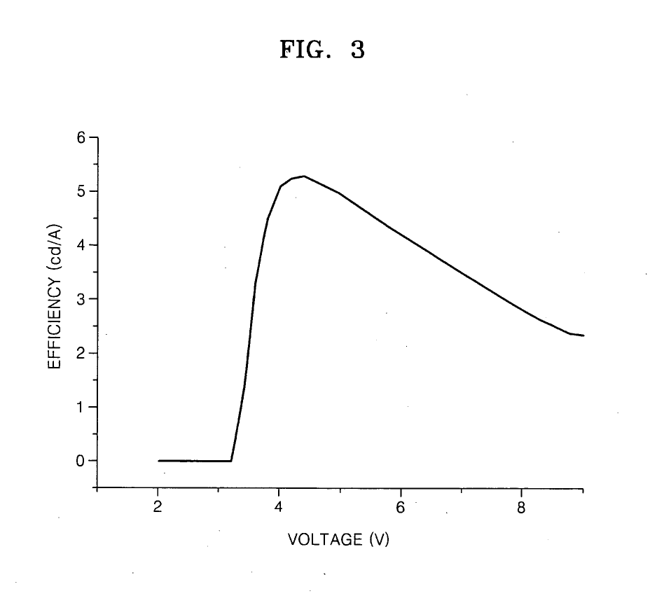

For the samples 1-4 and A, a driving voltage, brightness, and efficiency were evaluated using a PR650 (Spectroscan) Source Measurement Unit. The results are presented in Table 2 below. In particular, a voltage-efficiency graph of Compound 3 is shown in FIG. 3.

TABLE 2

| | Turn-on driving | Maximum efficiency | |
|---|---|---|---|
| Sample | voltage (V) | (cd/A) | Brightness (cd/m²) |
| 1 | 3.4 | 5.3 | 8210 |
| 2 | 3.4 | 4.9 | 7845 |
| 3 | 3.4 | 5.6 | 6798 |
| 4 | 3.8 | 3.1 | 5204 |
| A | 3.4 | 3.1 | 4500 |

Table 2 shows that the samples 1-4 according to the embodiments of the present invention have excellent electrical characteristics.

An aromatic compound of Formula 1 according to an embodiment of the present invention has excellent thermal stability and emission characteristics. Therefore, the use of the aromatic compound according to an embodiment of the present invention enables production of an organic light-emitting diode having a low driving voltage, high efficiency, and high brightness.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An aromatic compound represented by Formula 1:
M₁-(B)ₙ-M₂ (1)
wherein B is a single bond, a substituted or unsubstituted C₁-C₆₀ alkylene group, a substituted or unsubstituted C₅-C₆₀ cycloalkylene group, a substituted or unsubstituted C₅-C₆₀ heterocycloalkylene group, a substituted or unsubstituted C₅-C₆₀ arylene group, a substituted or unsubstituted C₂-C₆₀ heteroarylene group, or a divalent linking group represented by —N(Z₁)- where Z₁ is hydrogen, a substituted or unsubstituted C₁-C₆₀ alkyl group, or a substituted or unsubstituted C₅-C₆₀ aryl group;
n is an integer of 1 to 10; and
M₁ and M₂ are each independently a terminal group derived from a compound represented by Formula 2: 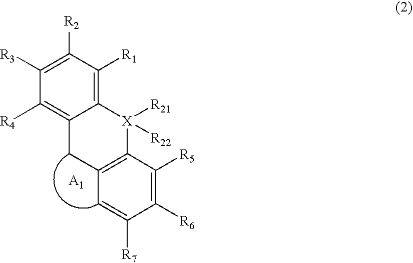
wherein X is a Group XIV element;
R₁, R₂, R₃, R₄, R₅, R₆, R₇, R₂₁, and R₂₂ are each independently hydrogen, halogen, a cyano group, an amino group, a nitro group, a hydroxyl group, a substituted or unsubstituted C₁-C₆₀ alkyl group, a substituted or unsubstituted C₁-C₆₀ alkoxy group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₂-C₆₀ heteroaryl group, a substituted or unsubstituted C₅-C₆₀ arylamino group, a substituted or unsubstituted C₁-C₆₀ alkylamino group, a substituted or unsubstituted C₅-C₆₀ arylsilyl group, or a substituted or unsubstituted C₁-C₆₀ alkylsilyl group, and two or more of R₁, R₂, R₃, R₄, R₅, R₆, R₇, R₂₁, and R₂₂ may be optionally connected or fused together to form a substituted or unsubstituted C₆-C₆₀ aromatic ring or a substituted or unsubstituted C₆-C₆₀ heteroaromatic ring; and
A₁ is a substituted or unsubstituted C₆-C₆₀ aromatic ring or a substituted or unsubstituted C₆-C₆₀ heteroaromatic ring.

2. The aromatic compound of claim 1, wherein the alkylene group, the cycloalkylene group, the heterocycloalkylene group, the arylene group, the heteroarylene group, the alkyl group, the alkoxy group, the alkenyl group, the alkynyl group, the cycloalkyl group, the cycloalkenyl group, the aryl group, and the heteroaryl group are substituted by at least one substituent selected from the group consisting of —F; —Cl; —Br; —CN; —NO₂; —NH₂; —OH; a C₁-C₆₀ alkyl group which is unsubstituted or substituted by a C₁-C₆₀ alkoxy group, —F, —Cl, —Br, —CN, —NO₂, —NH₂, or —OH; a C₅-C₆₀ cycloalkyl group which is unsubstituted or substituted by a C₁-C₆₀ alkyl group, a C₁-C₆₀ alkoxy group, —F, —Cl, —Br, —CN, —NO₂, —NH₂, or —OH; a C₅-C₆₀ aryl group which is unsubstituted or substituted by a C₁-C₆₀ alkyl group, a C₁-C₆₀ alkoxy group, —F, —Cl, —Br, —CN, —NO₂, —NH₂, or —OH; and a C₂-C₆₀ heteroaryl group which is unsubstituted or substituted by a C₁-C₆₀ alkyl group, a C₁-C₆₀ alkoxy group, —F, —Cl, —Br, —CN, —NO₂, —NH₂, or —OH.

3. The aromatic compound of claim 1, wherein B is a single bond, a substituted or unsubstituted C₁-C₁₀ alkylene group, a substituted or unsubstituted C₅-C₂₂ cycloalkylene group, a substituted or unsubstituted C₅-C₂₂ heterocycloalkylene group, a substituted or unsubstituted C₅-C₂₂ arylene group, a substituted or unsubstituted C₂-C₂₂ heteroarylene group, or a divalent linking group represented by —N(Z₁)- where Z₁ is hydrogen, a substituted or unsubstituted C₁-C₁₀ alkyl group, or a substituted or unsubstituted C₅-C₂₂ aryl group.

4. The aromatic compound of claim 1, wherein B is a single bond, an ethylene group, a propylene group, a cyclohexylene group, a phenylene group, a naphthylene group, a phenalenylene group, an anthracenylene group, a fluorenylene group, a pyridinylene group, a thiophenylene group, or a divalent linking group represented by —N(Z₁)- where Z₁ is a substituted or unsubstituted phenyl group.

5. The aromatic compound of claim 1, wherein n is 1, 2, 3, 4, or 5.

6. The aromatic compound of claim 1, wherein B is a single bond, or —(B)ₙ- is one of structures represented by Formulas 3a through 3v: 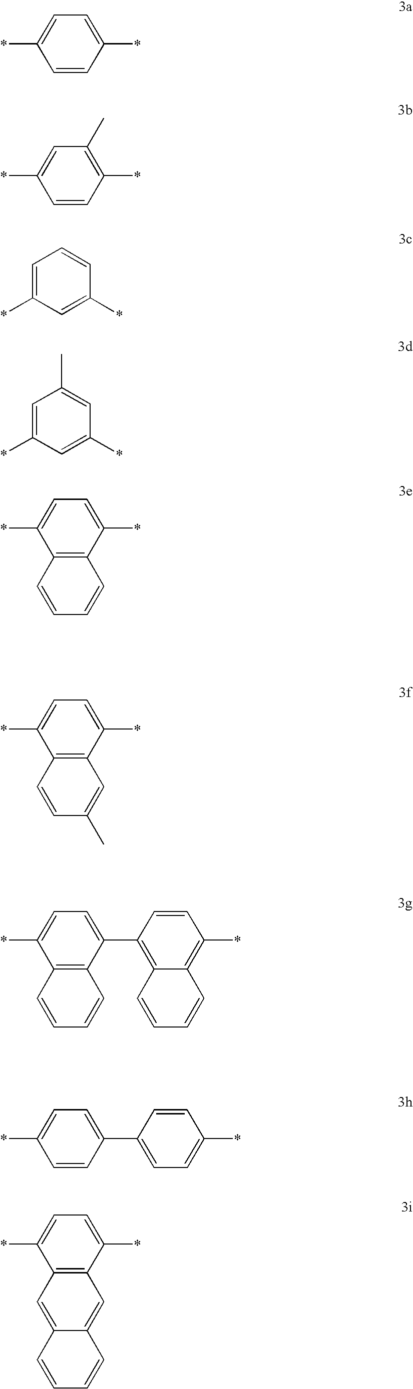 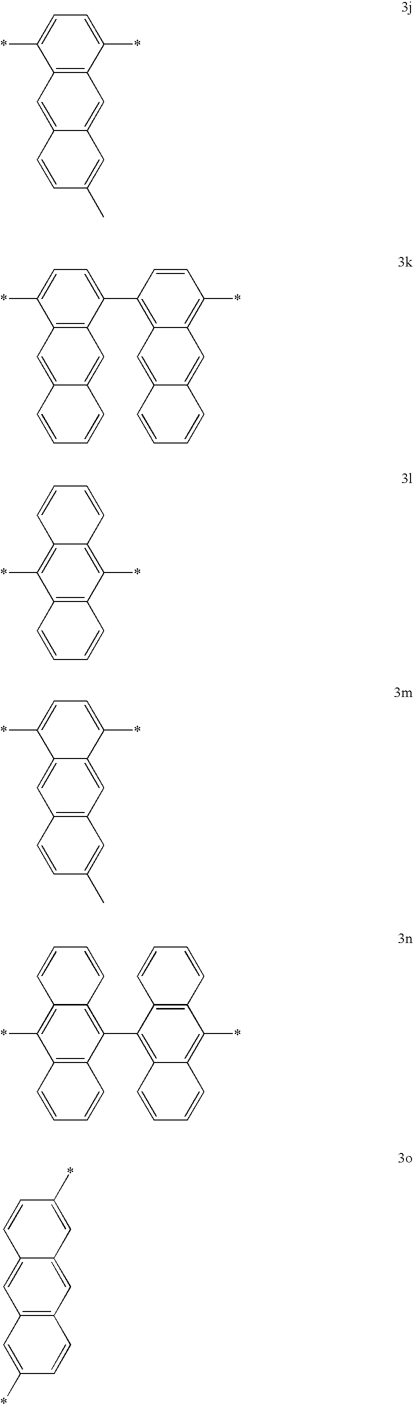 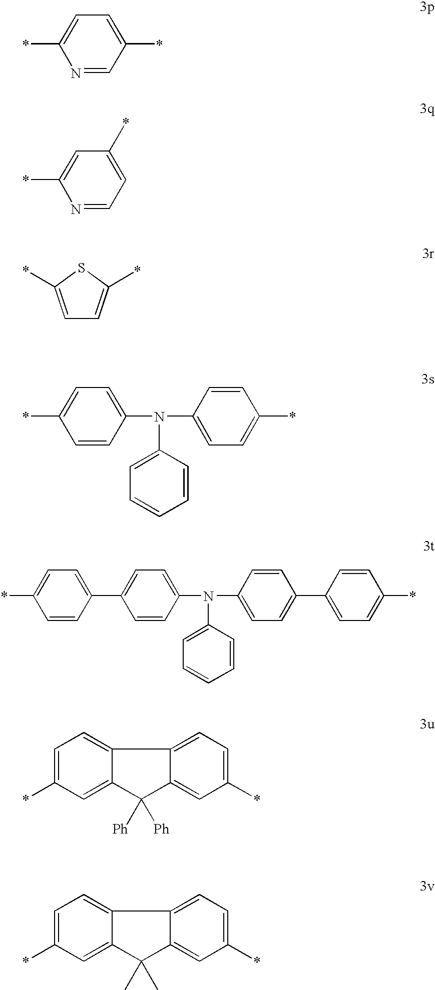
wherein two asterisks (*) of each structure respectively represent binding sites with M₁ and M₂, and Ph represents a phenyl group.

7. The aromatic compound of claim 1, wherein X is C, Si, or Ge.

8. The aromatic compound of claim 1, wherein A₁ is a substituted or unsubstituted benzene, a substituted or unsubstituted pentalene, a substituted or unsubstituted indene, a substituted or unsubstituted naphthalene, a substituted or unsubstituted azulene, a substituted or unsubstituted heptalene, a substituted or unsubstituted biphenylene, a substituted or unsubstituted indacene, a substituted or unsubstituted acenaphthylene, a substituted or unsubstituted fluorene, a substituted or unsubstituted phenalene, a substituted or unsubstituted phenanthrene, a substituted or unsubstituted anthracene, a substituted or unsubstituted fluoranthene, a substituted or unsubstituted acephenanthrylene, a substituted or unsubstituted aceanthrylene, a substituted or unsubstituted triphenylene, a substituted or unsubstituted pyrene, a substituted or unsubstituted chrysene, a substituted or unsubstituted naphthacene, a substituted or unsubstituted picene, a substituted or unsubstituted perylene, a substituted or unsubstituted pentaphene, a substituted or unsubstituted pentacene, a substituted or unsubstituted tetraphenylene, a substituted or unsubstituted hexaphene, a substituted or unsubstituted hexacene, a substituted or unsubstituted rubicene, a substituted or unsubstituted coronene, a substituted or unsubstituted pyranthrene, a substituted or unsubstituted ovalene, a substituted or unsubstituted thiophene, a substituted or unsubstituted indole, a substituted or unsubstituted furan, a substituted or unsubstituted benzothiophene, a substituted or unsubstituted parathiazine, a substituted or unsubstituted benzofuran, a substituted or unsubstituted pyrrole, a substituted or unsubstituted pyrazole, a substituted or unsubstituted imidazole, a substituted or unsubstituted imidazoline, a substituted or unsubstituted oxazole, a substituted or unsubstituted thiazole, a substituted or unsubstituted triazole, a substituted or unsubstituted tetrazole, a substituted or unsubstituted oxadiazole, a substituted or unsubstituted pyridine, a substituted or unsubstituted pyridazine, a substituted or unsubstituted pyrazine, a substituted or unsubstituted pyrimidine, a substituted or unsubstituted indole, a substituted or unsubstituted benzimidazole, a substituted or unsubstituted quinoline, a substituted or unsubstituted phenothiazine, or a substituted or unsubstituted thianthrene.

9. The aromatic compound of claim 1, wherein R₁, R₂, R₃, R₄, R₅, R₆, R₇, R₂₁, and R₂₂ are each independently selected from the group consisting of hydrogen, a C₁-C₆₀ alkyl group, a C₂-C₆₀ alkenyl group, a C₂-C₆₀ alkynyl group, a C₅-C₆₀ cycloalkyl group, a C₅-C₆₀ cycloalkenyl group, a C₅-C₆₀ cycloalkynyl group, a cyclohexyl group, a phenyl group, a biphenyl group, a pentalenyl group, an indenyl group, a naphthyl group, a biphenylenyl group, an anthracenyl group, an azulenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxiranyl group, a pyrrolidinyl group, a pyrazolidinyl group, an imidazolidinyl group, a piperidinyl group, a piperazinyl group, a morpholinyl group, a di(C₅-C₆₀ aryl)amino group, a tri(C₅-C₆₀ alkyl)silyl group, a tri(C₅-C₆₀ aryl)silyl group, a diphenylaminophenyl group, a ditolylaminophenyl group, and derivatives thereof.

10. The aromatic compound of claim 1, wherein R₁, R₂, R₃, R₄, R₅, R₆, and R₇ are each independently selected from the group consisting of hydrogen, a methyl group, a cyclohexyl group, a phenyl group, a biphenyl group, a tolyl group, a naphthyl group, a pyrenyl group, a phenanthrenyl group, a fluorenyl group, an imidazolinyl group, an indolyl group, a quinolinyl group, a diphenylamino group, a N,N-diphenylaminophenyl group, a N,N-di-p-tolylaminophenyl group, a trimethylsilyl group, a triphenylsilyl group, and derivatives thereof.

11. The aromatic compound of claim 1, wherein R₂₁ and R₂₂ are each independently hydrogen, —CH₃, —C₆H₁₁, or a phenyl group.

12. The aromatic compound of claim 1, wherein the compound of Formula 2 is a compound represented by Formula 2a, 2b, or 2c: 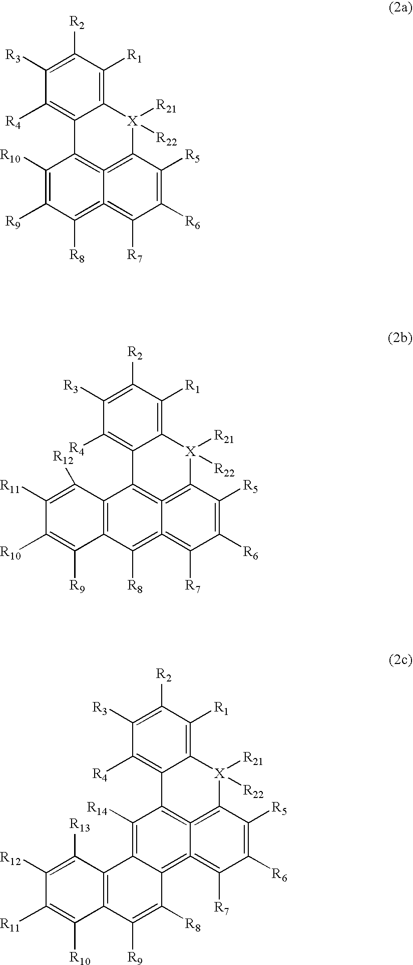
wherein X is a Group XIV element;
R₁, R₂, R₃, R₄, R₅, R₆, R₇, R₈, R₉, R₁₀, R₁₁, R₁₂, R₁₃, R₁₄, R₂₁, and R₂₂ are each independently hydrogen, halogen, a cyano group, an amino group, a nitro group, a hydroxyl group, a substituted or unsubstituted C₁-C₆₀ alkyl group, a substituted or unsubstituted C₁-C₆₀ alkoxy group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₂-C₆₀ heteroaryl group, a substituted or unsubstituted C₅-C₆₀ arylamino group, a substituted or unsubstituted C₁-C₆₀ alkylamino group, a substituted or unsubstituted C₅-C₆₀ arylsilyl group, or a substituted or unsubstituted C₁-C₆₀ alkylsilyl group, and two or more of R₁, R₂, R₃, R₄, R₅, R₆, R₇, R₈, R₉, R₁₀, R₁₁, R₁₂, R₁₃, R₁₄, R₂₁, and R₂₂ may be optionally connected or fused together to form a substituted or unsubstituted C₆-C₆₀ aromatic ring or a substituted or unsubstituted C₆-C₆₀ heteroaromatic ring.

13. The aromatic compound of claim 1, wherein the terminal group derived from the compound of Formula 2 is one of structures represented by Formulas 4a through 4u: 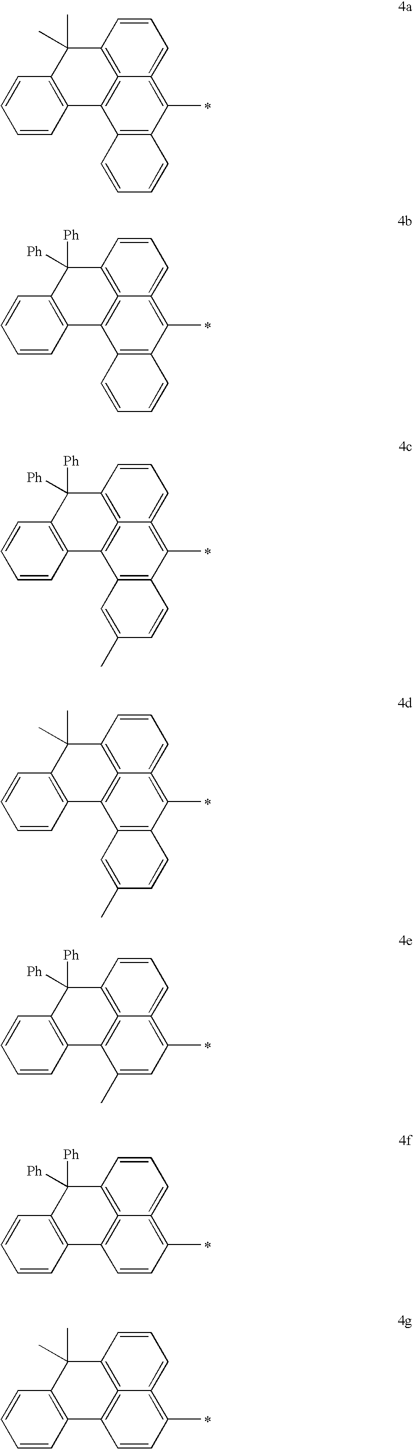 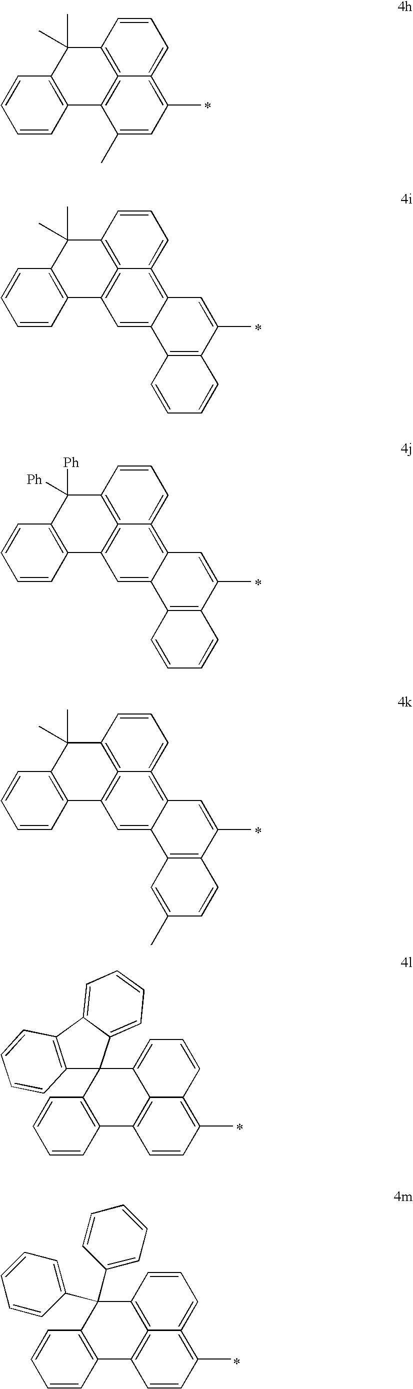 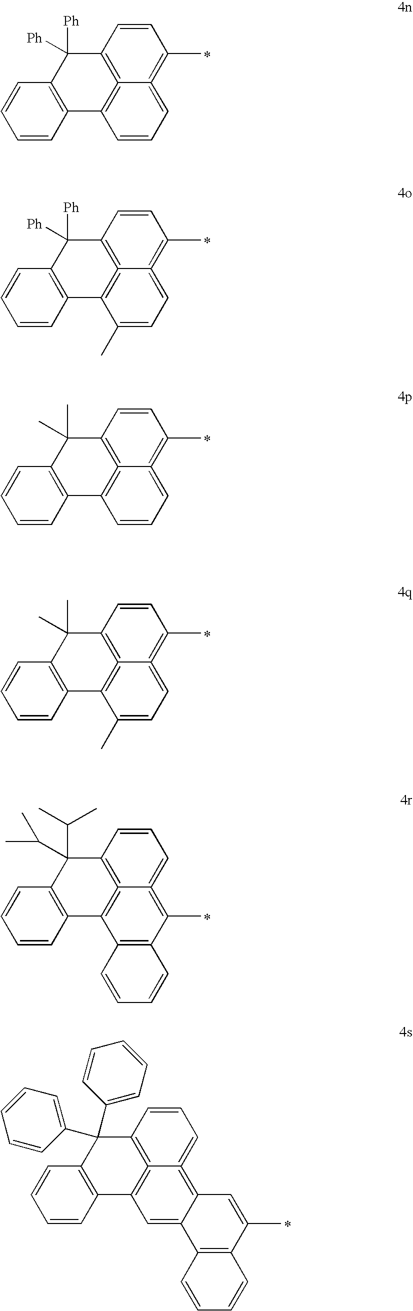 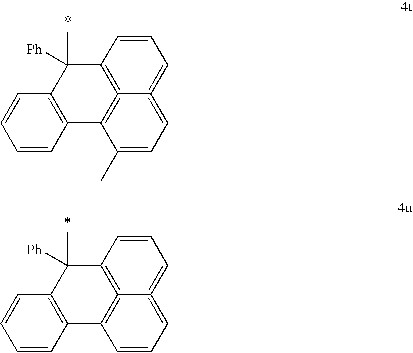
wherein an asterisk (*) of each structure represents a binding site with B, and Ph represents a phenyl group.

14. The aromatic compound of claim 1, which is one of compounds represented by Formulas 5 through 30:

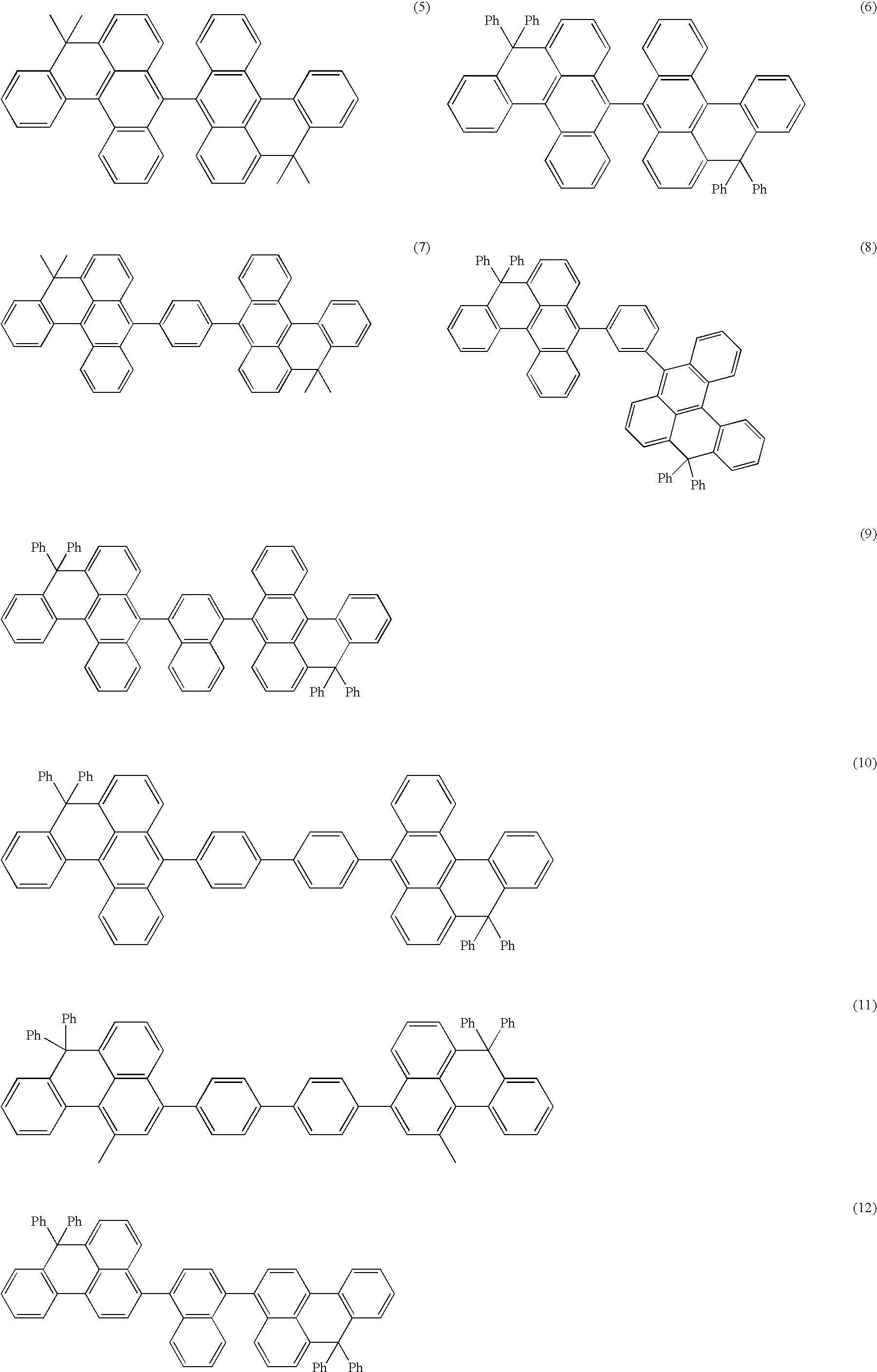

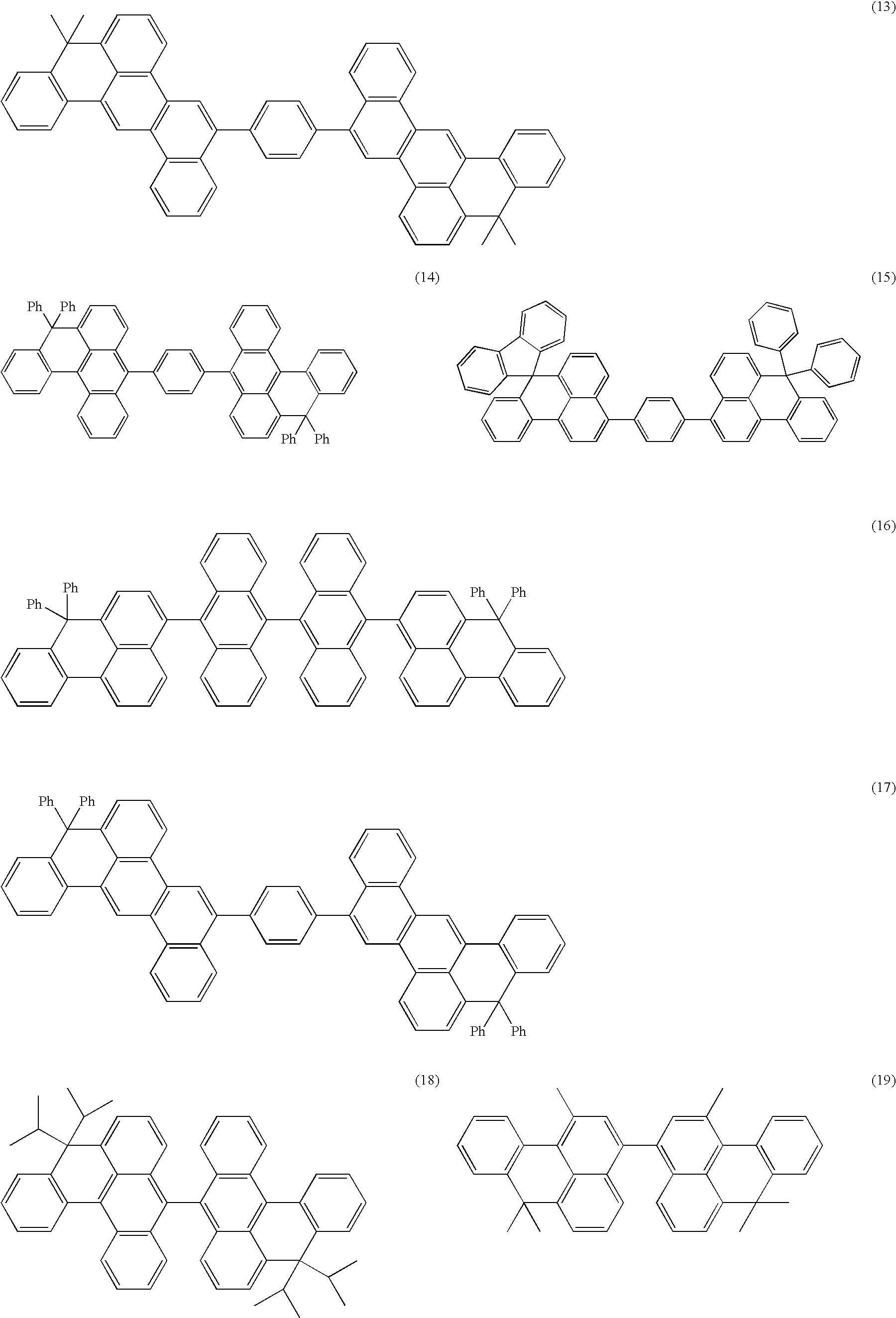

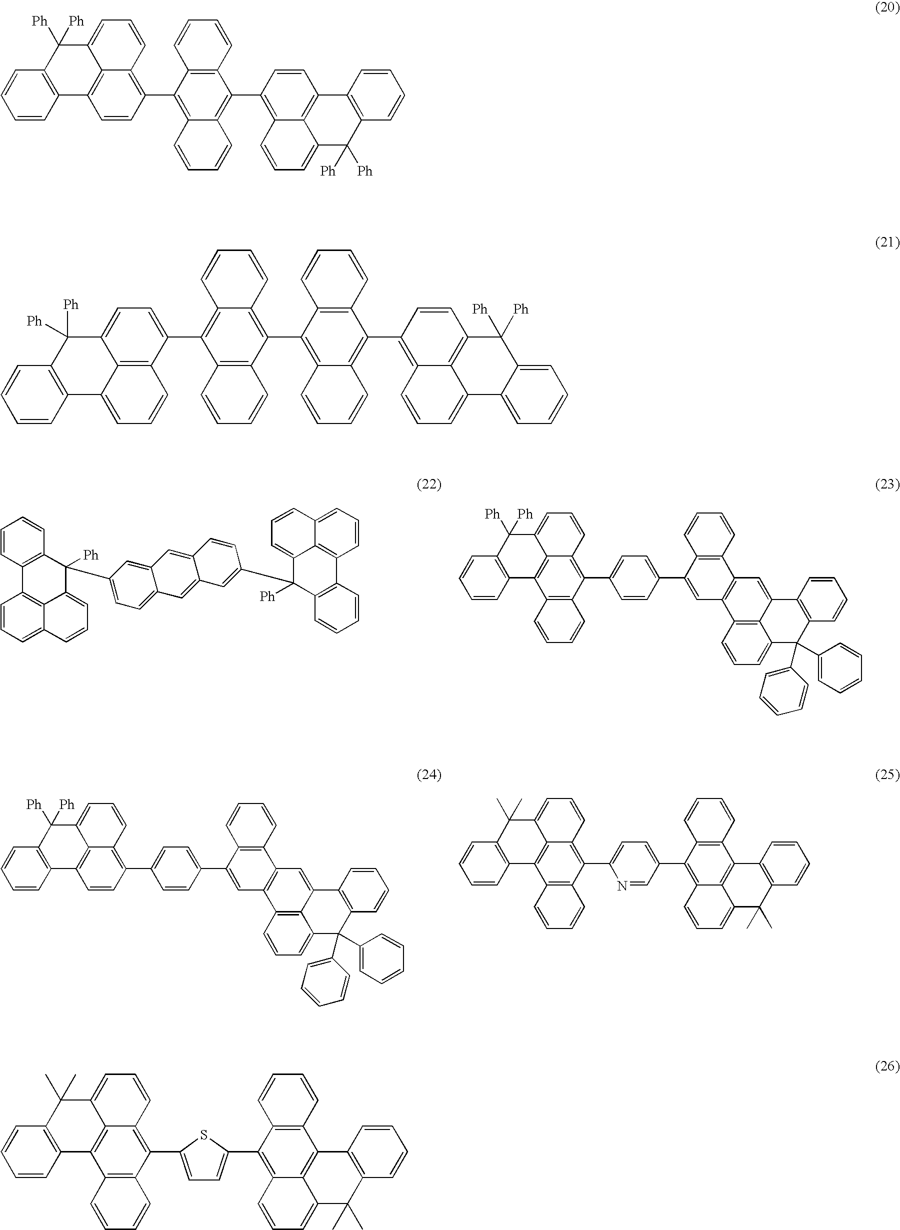

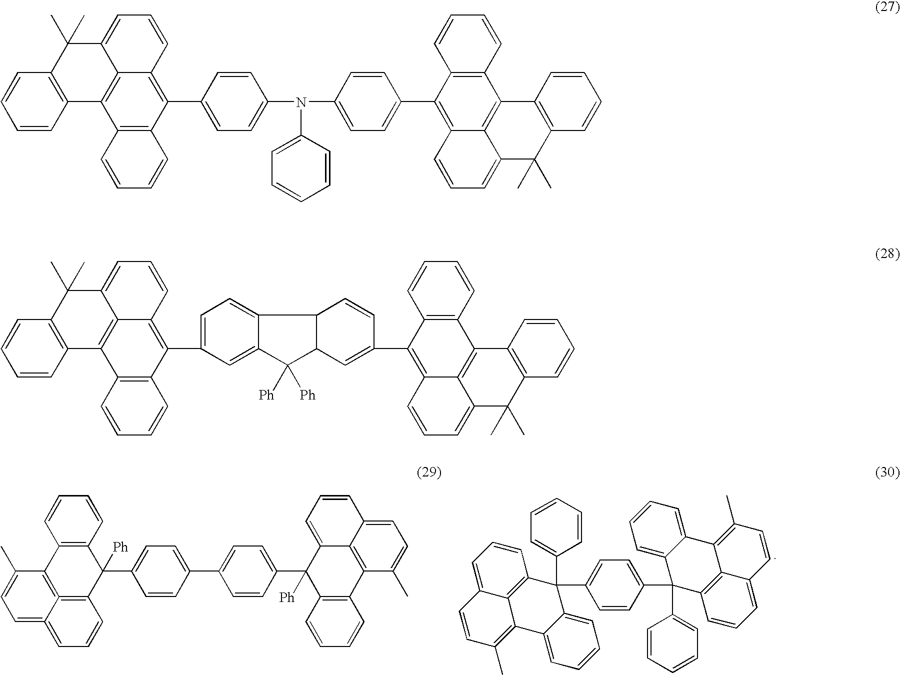

15. The aromatic compound of claim 1, which is one of compounds represented by Formulas 7, 14, 17 and 30:

16. An organic light-emitting diode comprising:
a first electrode;
a second electrode; and
an organic layer interposed between the first electrode and the second electrode, the organic layer including an aromatic compound represented by Formula 1:
M₁-(B)ₙ-M₂ (1)
wherein B is a single bond, a substituted or unsubstituted C₁-C₆₀ alkylene group, a substituted or unsubstituted C₅-C₆₀ cycloalkylene group, a substituted or unsubstituted C₅-C₆₀ heterocycloalkylene group, a substituted or unsubstituted C₅-C₆₀ arylene group, a substituted or unsubstituted C₂-C₆₀ heteroarylene group, or a divalent linking group represented by —N(Z₁)- where Z₁ is hydrogen, a substituted or unsubstituted C₁-C₆₀ alkyl group, or a substituted or unsubstituted C₅-C₆₀ aryl group;
n is an integer of 1 to 10; and
M₁ and M₂ are each independently a terminal group derived from a compound represented by Formula 2: 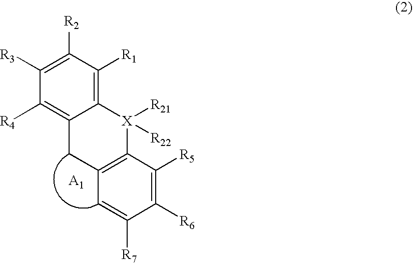
wherein X is a Group XIV element;
R₁, R₂, R₃, R₄, R₅, R₆, R₇, R₂₁, and R₂₂ are each independently hydrogen, halogen, a cyano group, an amino group, a nitro group, a hydroxyl group, a substituted or unsubstituted C₁-C₆₀ alkyl group, a substituted or unsubstituted C₁-C₆₀ alkoxy group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₂-C₆₀ heteroaryl group, a substituted or unsubstituted C₅-C₆₀ arylamino group, a substituted or unsubstituted C₁-C₆₀ alkylamino group, a substituted or unsubstituted C₅-C₆₀ arylsilyl group, or a substituted or unsubstituted C₁-C₆₀ alkylsilyl group, and two or more of R₁, R₂, R₃, R₄, R₅, R₆, R₇, R₂₁, and R₂₂ may be optionally connected or fused together to form a substituted or unsubstituted C₆-C₆₀ aromatic ring or a substituted or unsubstituted C₆-C₆₀ heteroaromatic ring; and
A₁ is a substituted or unsubstituted C₆-C₆₀ aromatic ring or a substituted or unsubstituted C₆-C₆₀ heteroaromatic ring.

17. The organic light-emitting diode of claim 16, wherein the organic layer including an aromatic compound is a light-emitting layer, a hole injection layer, a hole transport layer, a hole blocking layer, or an electron transport layer.

18. The organic light-emitting diode of claim 16, further comprising at least one layer selected from the group consisting of a hole injection layer, a hole transport layer, a hole blocking layer, an electron transport layer, and an electron injection layer, between the first electrode and the second electrode.

19. A method of manufacturing an organic light-emitting diode, the method comprising:
forming a first electrode on a substrate;
forming on the first electrode an organic layer including an aromatic compound represented by Formula 1:
M₁-(B)ₙ-M₂ (1)
wherein B is a single bond, a substituted or unsubstituted C₁-C₆₀ alkylene group, a substituted or unsubstituted C₅-C₆₀ cycloalkylene group, a substituted or unsubstituted C₅-C₆₀ heterocycloalkylene group, a substituted or unsubstituted C₅-C₆₀ arylene group, a substituted or unsubstituted C₂-C₆₀ heteroarylene group, or a divalent linking group represented by —N(Z₁)- where Z₁ is hydrogen, a substituted or unsubstituted C₁-C₆₀ alkyl group, or a substituted or unsubstituted C₅-C₆₀ aryl group;
n is an integer of 1 to 10; and
M₁ and M₂ are each independently a terminal group derived from a compound represented by Formula 2: 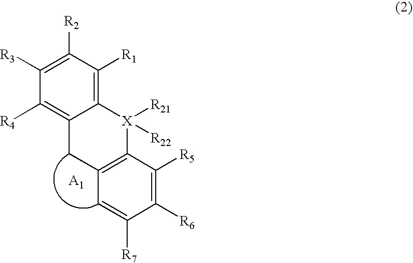
wherein X is a Group XIV element;
R₁, R₂, R₃, R₄, R₅, R₆, R₇, R₂₁, and R₂₂ are each independently hydrogen, halogen, a cyano group, an amino group, a nitro group, a hydroxyl group, a substituted or unsubstituted C₁-C₆₀ alkyl group, a substituted or unsubstituted C₁-C₆₀ alkoxy group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₂-C₆₀ heteroaryl group, a substituted or unsubstituted C₅-C₆₀ arylamino group, a substituted or unsubstituted C₁-C₆₀ alkylamino group, a substituted or unsubstituted C₅-C₆₀ arylsilyl group, or a substituted or unsubstituted C₁-C₆₀ alkylsilyl group, and two or more of R₁, R₂, R₃, R₄, R₅, R₆, R₇, R₂₁, and R₂₂ may be optionally connected or fused together to form a substituted or unsubstituted C₆-C₆₀ aromatic ring or a substituted or unsubstituted C₆-C₆₀ heteroaromatic ring; and
A₁ is a substituted or unsubstituted C₆-C₆₀ aromatic ring or a substituted or unsubstituted C₆-C₆₀ heteroaromatic ring; and
forming a second electrode on the organic layer.

20. The method of claim 19, wherein the formation of the organic layer is performed using a vacuum deposition process, a spin coating process, an inkjet printing process, a screen printing process, a spray printing process, or a thermal transfer process.
